(19) United States
(12) Patent Application Publication
(10) Pub. No.: US 2003/0108540 A1
(43) Pub. Date: Jun. 12, 2003

(54) Anti-angiogenic and anti-tumor properties of matin and other laminin domains

(75) Inventor: Raghuram Kalluri, Weston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Beth Israel Deaconess Medical Center, Boston, MA

(21) Appl. No.: 10/262,670

(22) Filed: Sep. 30, 2002

Related U.S. Application Data
(63) Continuation-in-part of application No. PCT/US01/09921, filed on Mar. 28, 2001.
(60) Provisional application No. 60/192,875, filed on Mar. 29, 2000.

(51) Int. Cl. A61K 38/48; C12N 9/64

(52) U.S. Cl. 424/94.63; 435/226

(57) ABSTRACT

A protein with anti-angiogenic properties is disclosed.

### RELATED APPLICATIONS

This application is a continuation-in-part of International Application No. PCT/US01/09921, which designated the United States and was filed Mar. 28, 2001, published in English, which claims the benefit of U.S. Provisional Application No. 60/192,875, filed on Mar. 29, 2000.

The entire teachings of the above applications are incorporated herein by reference.

### BACKGROUND OF THE INVENTION

Basement membranes are thin layers of specialized extracellular matrix that provide supporting structure on which epithelial and endothelial cells grow, and that surround muscle or fat (Paulsson, M., 1992, Crit. Rev. Biochem. Mol. Biol. 27:93-127). Basement membranes are always associated with cells, and it has been well documented that basement membranes not only provide mechanical support, but also influence cellular behavior such as differentiation and proliferation. Vascular basement membranes are composed of macromolecules such as collagen, laminin, heparan sulfate proteoglycans, fibronectin and nidogen (also called entactin) (Timpl, R., 1996, Curr. Opin. Cell. Biol. 8:618-624).

Angiogenesis is the process of formation of new blood vessels from pre-existing ones (Madri, J. A. et al., 1986, J. Histochem. Cytochem. 34:85-91; Folkman, J., 1972, Ann. Surg. 175:409-416). Angiogenesis is a complex process, and requires sprouting and migration of endothelial cells, proliferation of those cells, and their differentiation into tube-like structures and the production of a basement membrane matrix around the developing blood vessel. Additionally angiogenesis is a process critical for normal physiological events such as wound repair and endometrium remodeling (Folkinan, J. et al., 1995, J. Biol. Chem. 267:10931-10934). It is now well documented that angiogenesis is required for metastasis and growth of solid tumors beyond a few mm³ in size (Folkman, J., 1972, Ann. Surg. 175:409-416; Folkman, J., 1995, Nat. Med. 1:27-31). Expansion of tumor mass occurs not only by perfusion of blood through the tumor, but also by paracrine stimulation of tumor cells by several growth factors and matrix proteins produced by the new capillary endothelium (Folkman, J., 1995, Nat. Med. 1:27-31). Recently, a number of angiogenesis inhibitors have been identified, namely angiostatin (O'Reilly, M. S. et al., 1994, Cell 79:315-28), endostatin (O'Reilly, M. S. et al., 1997, Cell 88:277-85), restin (Ramchandran, R. et al., 1999, Biochem. Biophys. Res. Commun. 255:735-9), Arresten (Colorado, P. C. et al., 2000, Cancer Res. 60:2520-6), Canstatin (Kamphaus, G. D. et al., 2000, J. Biol. Chem. 275:1209-15) and Tumstatin (Maeshima, Y. et al., 2000, J. Biol. Chem. 275:21340-8; Maeshima, Y. et al., 2000, J Biol. Chem. 275:23745-50) and pigment epithelium-derived factor (PEDF) (Dawson, D. W. et al., 1999, Science 285:245-8).

### SUMMARY OF THE INVENTION

The present invention relates to isolated proteins or polypeptides comprising laminin, and fragments, mutants, analogs, homologs and derivatives thereof. In particular, a laminin protein, fragment, mutant, analog, homolog or derivative thereof has at least one anti-angiogenic property or anti-tumor activity. Laminin fragments, mutants, analogs, homologs and derivatives having at least one anti-angiogenic property or anti-tumor activity can comprise α, β₁, or β₂ chains of laminin, or a combination or a permutation of α, β₁, or β₂ chains of laminin, or fragments, mutants, analogs, homologs and derivatives thereof. Furthermore, a laminin protein, fragment, mutant, analog, homolog or derivative thereof, having at least one anti-angiogenic property or anti-tumor activity, can be multimer (e.g. dimer, trimer, etc.) or a fusion protein (also referred to herein as a chimeric protein) thereof.

The present invention also relates to proteins comprising the globular domains of the α1 chain of laminin having anti-angiogenic properties. In particular, the present invention relates to the novel protein designated herein as “Matin,” which comprises the G1 domain of laminin, and to biologically active (e.g., anti-angiogenic) fragments, mutants, analogs, homologs and derivatives thereof, as well as multimers (e.g., dimers, trimers, etc.) and fusion proteins thereof. In particular, Matin is a monomeric protein, and arrests endothelial cell proliferation in vivo.

In particular, the invention features isolated Matin, where Matin is an isolated protein of the G1 domain of the α1 chain of laminin, or a fragment, analog, derivative or mutant thereof, where the protein, fragment, analog, derivative or mutant thereof has anti-angiogenic activity. The Matin can be the G1 domain of the α1 chain of another laminin, other globular domains, globular domains from other α chains, other laminins, laminins from other mammals, and fragments, mutants, homologs, analogs and allelic variants of the Matin amino acid sequence. The Matin, or a fragment, analog, derivative or mutant thereof, can be a monomer, a multimer, or a chimeric protein, having anti-angiogenic or anti-tumor activity. The Matin can be about 28 to about 32 kDa in size, or about 30 kDa in size.

The invention also features the isolated protein of about residue 2352 to about residue 2354 of SEQ ID NO: 2, about residue 2352 to about residue 2553 of SEQ ID NO: 2, about residue 2534 to about residue 2761 of SEQ ID NO: 2, about residue 2762 to about residue 2895 of SEQ ID NO: 2, about residue 2896 to about residue 3100 of SEQ ID NO: 2, or a fragment, analog, derivative or mutant thereof, where the protein, fragment, analog, derivative or mutant has anti-angiogenic activity. The protein, or a fragment, analog, derivative or mutant thereof, can be a monomer, dimer, trimer, multimer, or a chimeric protein, having anti-angiogenic or anti-tumor activity. The proteins, fragments, etc., from the other G domains of laminin are alternative sources of Matin.

The invention also features an isolated polynucleotide encoding a laminin protein, or a fragment, analog, homolog, derivative or mutant of a laminin protein having at least one anti-angiogenic property or anti-tumor activity. The isolated polynucleotide sequence can be operably-linked to an expression control sequence. The polynucleotide can be used (with or without an operable linkage to an expression control sequence) to transform a host cell. The host cell can be selected from the group comprising bacterial, yeast, mammalian, insect or plant cells.

The invention also features an isolated protein or peptide having 90% or greater sequence identity with SEQ ID NO: 2, where the protein or peptide has anti-angiogenic activity, and an isolated protein or peptide having 85% or greater sequence identity with SEQ ID NO: 2, where the protein or peptide has anti-angiogenic activity.

The invention also features an isolated polynucleotide having 90% or greater sequence identity with SEQ ID NO: 1, where the polynucleotide encodes a protein having anti-angiogenic activity.

The chimeric protein described above can further comprise at least one protein molecule selected from the group consisting of: Vascostatin or fragments thereof, arresten or fragments thereof, canstatin or fragments thereof, tumstatin or fragments thereof, endostatin or fragments thereof, angiostatin or fragments thereof, restin or fragments thereof, apomigren or fragments thereof, or other anti-angiogenic proteins or fragments thereof. Vascostatin comprises the C-terminal globular domain of nidogen, and has anti-angiogenic properties. Vascostatin is described in International application PCT/US01/40382, “Anti-Angiogenic and Anti-tumor Properties of Vascostatin and Other Nidogen Domains”, by Raghuram Kalluri, filed Mar. 28, 2001.

The invention further features a composition comprising, as a biologically active ingredient, one or more of the proteins, fragments, analogs, derivatives, mutants, monomers, multimers or chimeric proteins described above. The composition may also include a pharmaceutically-compatible carrier. The composition may further comprise at least one protein molecule selected from the group consisting of: Vascostatin or fragments thereof, arresten or fragments thereof, canstatin or fragments thereof, tumstatin or fragments thereof, endostatin or fragments thereof, angiostatin or fragments thereof, restin or fragments thereof, apomigren or fragments thereof, or other anti-angiogenic proteins, or fragments thereof. The composition may be used in a method of inhibiting a disease characterized by angiogenic activity, where the method comprises administering to a patient with the disease, the composition in conjunction with radiation therapy, chemotherapy, or immunotherapy.

In another aspect, the invention features a process for producing a protein encoded by the polynucleotide described above, where the process comprises: (a) growing a culture of a host cell transformed with the polynucleotide described above, where the host cell is selected from the group comprising bacterial, yeast, mammalian, insect or plant cells; and (b) purifying the protein from the culture, so that the protein encoded by the polynucleotide described above is produced.

The invention also features an isolated polynucleotide produced according to the process of: (a) preparing one or more polynucleotide probes that hybridize under conditions under moderate stringency to the polynucleotide described above; (b) hybridizing the probe(s) to mammalian DNA; and (c) isolating the DNA polynucleotide detected with the probe(s); so that the nucleotide sequence of the isolated polynucleotide corresponds to the nucleotide sequence of the polynucleotide described above.

The invention further features a process for providing a mammal with an anti-angiogenic protein, where the process comprises introducing mammalian cells into a mammal, the mammalian cells having been treated in vitro to insert within them the polynucleotide described above, and where the mammalian cell express in vivo within the mammal a therapeutically effective amount of the anti-angiogenic protein in an amount sufficient to inhibit angiogenic activity in the mammal. The expression of the anti-angiogenic protein may be transient or permanent expression. The mammalian cells may be chosen from the group consisting of: blood cells, TIL cells, bone marrow cells, vascular cells, tumor cells, liver cells, muscle cells, fibroblast cells. The polynucleotide may be inserted into the cells by a viral vector.

The invention additionally features antibodies that specifically bind to the isolated Matin protein, fragment, analog, derivative or mutant, or the Matin monomers, dimer, trimers, multimers or chimeric proteins described above.

In another aspect, the invention features a method for inhibiting angiogenic activity in mammalian tissue, where the method comprises contacting the tissue with a composition comprising one or more of the following: the Matin protein, fragment, analog, derivative or mutant described above, or the Matin monomers, multimers or chimeric proteins as described above. The angiogenic activity may be characteristic of a disease selected from the group comprising angiogenesis-dependent cancers, benign tumors, rheumatoid arthritis, diabetic retinopathy, psoriasis, ocular angiogenesis diseases, Osler-Webber Syndrome, myocardial angiogenesis, plaque neovascularization, telangiectasia, hemopheliac joints, angiofibroma, wound granulation, intestinal adhesions, atherosclerosis, scleroderna, hypertrophic scars, cat scratch disease, Heliobacter pylori ulcers, dialysis graft vascular access stenosis, contraception and obesity. The disease may be cancer.

The invention further features an isolated polynucleotide encoding an anti-angiogenic protein, where the isolated polynucleotide is produced by the process of: (a) preparing one or more polynucleotide probes that hybridize under conditions under moderate to high stringency to nucleotide 6442 to nucleotide 7062 of SEQ ID NO: 1; (b) hybridizing the probe(s) to mammalian DNA; and (c) isolating the polynucleotide detected with the probe(s); so that the nucleotide sequence of the isolated polynucleotide has anti-angiogenic activity and corresponds to the nucleotide sequence of nucleotide 6442 to nucleotide 7062 of SEQ ID NO: 1. The probes may be SEQ ID NO: 3 and SEQ ID NO: 4. The isolated polynucleotide may also be a subsequence of SEQ ID NO: 1. Alternatively, the polynucleotide can also correspond to about nucleotide 7054 to about nucleotide 7599, nucleotide 7600 to about nucleotide 8283, nucleotide 8284 to about nucleotide 8685, or nucleotide 8686 to about nucleotide 9300.

The invention also features a method for producing an anti-angiogenic polypeptide, where the method comprises: (a) growing a culture of a host cell transformed with the polynucleotide of nucleotide 6442 to nucleotide 7062 of SEQ ID NO: 1, where the host cell is selected from the group comprising bacterial, yeast, mammalian, insect or plant cells; and (b) purifying the protein from the culture; so that an anti-angiogenic polypeptide is produced. Alternatively, the polynucleotide can also correspond to about nucleotide 7054 to about nucleotide 7599, nucleotide 7600 to about nucleotide 78283, nucleotide 8284 to about nucleotide 8685, or nucleotide 8686 to about nucleotide 9300.

### BRIEF DESCRIPTION OF THE DRAWINGS

FIGS. 1A, 1B, 1C, 1D, 1E, 1F, 1G, 1H, 1I and 1J. are a diagram depicting the nucleotide (SEQ ID NO: 1) and amino acid (SEQ ID NO: 2) sequences of the five globular domains (G1, G2, G3, G4 and G5) of the oal chain of laminin-1 (Gen-Bank Acc.No. NM_{—}008480). Forward primers are depicted in bold face type with single underlining, and reverse primers are shown with double underlining. Globular domain 1 (G1) extends from about nucleotide 6442 to about nucleotide 7062, and about amino acid 2132 to about amino acid 2338. The forward primer used for the G1 domain was 5′-CGG-GAT-CCT-AGA-GAC-TGC-ATC-CGC-GCC-TAT-3′ (SEQ ID NO: 3), and the reverse primer was 5′-CCC-AAG-CTT-TAC-TAT-CTG-CGT-CAC-GGT-GGG-3′ (SEQ ID NO: 4). Globular domain 2 (G2) extends from about nucleotide 7054 to about nucleotide 7599, and about amino acid 2336 to about amino acid 2517. The forward primer used for the G2 domain was 5′-CGG-GAT-CCT-CAG-ATA-GTA-ATT-CTC-TTC-AGC-ACC-3′ (SEQ ID NO: 5), and the reverse primer was 5′-CCC-AAG-CTT-GGA-TGA-CTC-AGG-TGA-GAG-AGA-3′ (SEQ ID NO: 6). Globular domain 3 (G3) extends from about nucleotide 7600 to about nucleotide 8283, and about amino acid 2518 to about amino acid 2745. The forward primer used for the G3 domain was 5′-CGG-GAT-CCT-CTG-CTG-GCC-ACA-TTC-GCC-A-3′ (SEQ ID NO: 7), and the reverse primer was 5′-CCC-AAG-CTT-CCT-CTT-CCG-GAC-ATC-AGA-C-3′ (SEQ ID NO: 8). Globular domain 4 (G4) extends from about nucleotide 8284 to about nucleotide 8685, and about amino acid 2746 to about amino acid 2879. The forward primer used for the G4 domain was 5′-CGG-GAT-CCT-CTC-CAG-GTG-CAG-CTG-AGC-ATT-3′ (SEQ ID NO: 9), and the reverse primer was 5′-CCC-AAG-CTT-CTG-TTG-GCC-ATT-AAC-CAT-GAT-3′ (SEQ ID NO: 10). Globular domain 5 (G5) extends from about nucleotide 8686 to about nucleotide 9300, and about amino acid 2880 to about amino acid 3084. The forward primer used for the G5 domain was 5′-CGG-GAT-CCT-CTG GAT-AAA-GAC-AGG-CCC-TTG-3′ (SEQ ID NO: 11), and the reverse primer was 5′-CCC-AAG-CTT-GGG-CTC-AGG-CCC-GGG-GCA-GGA-AT-3′ (SEQ ID NO: 12). Underlined portions of the above primers correspond to the laminin sequence.

FIG. 2 is a schematic diagram representing the Matin cloning vector pET22b(+). Forward (SEQ ID NO: 3) and reverse (SEQ ID NO: 4) primers and site into which Matin was cloned are indicated.

FIGS. 3A and 3B are histograms showing the effect of varying concentrations of Matin (x-axis) on proliferation of endothelial (C-PAE) cells (FIG. 3A) and non-endothelial (PC-3) cells (FIG. 3B). Proliferation was measured as a function of methylene blue staining.

FIG. 4 is a plot showing annexin V fluorescence for cells treated with Matin as compared to controls.

FIGS. 5A and 5B are a pair of bar charts showing caspase-3 activity in Matin-treated CPAE cells (FIG. 5A) as compared to PC-3 cells (FIG. 5B).

FIGS. 6A and 6B are a pair of histograms showing cell viability at increasing concentrations of Matin (x-axis) as a function of OD₅₉₀ (y-axis) in an MTT apoptosis assay for CPAE cells (FIG. 6A) as compared to PC-3 cells (FIG. 6B). Each point represents the mean +/− the standard error of the mean for triplicate wells.

FIG. 7 is a line graph showing the effect on tumor size (mm³, y-axis) against days of treatment (x-axis) with 20 mg/ml Matin (▪) versus controls (20 mg/ml nephrin (◯) and PBS (□).

### DETAILED DESCRIPTION OF THE INVENTION

A wide variety of diseases are the result of undesirable angiogenesis. Put another way, many diseases and undesirable conditions could be prevented or alleviated if it were possible to stop the growth and extension of capillary blood vessels under some conditions, at certain times, or in particular tissues.

The formation of new capillaries from pre-existing vessels, angiogenesis, is essential for the process of tumor growth and metastasis (Folkman, J. et al., 1992, J. Biol. Chem. 267:10931-10934; Folkman, J., 1995, Nat. Med. 1:27-31; Hanahan, D. et al., 1996, Cell 86:353-364). Human and animal tumors are not vascularized at the beginning, however for a tumor to grow beyond few mm³, it might vascularize (Folkman, J., 1995, Nat. Med. 1:27-31; Hanahan, D. et al., 1996, Cell 86:353-364). The switch to an angiogenic phenotype requires both upregulation of angiogenic stimulators and downregulation of angiogenesis inhibitors (Folkman, J., 1995, Nat. Med. 1:27-31). Vascular endothelial growth factor (VEGF) and basic fibroblast growth factor (bFGF) are the most commonly expressed angiogenic factors in tumors. Vascularized tumors may overexpress one or more of these angiogenic factors which can synergistically promote tumor growth. Inhibition of a single angiogenic factor such as VEGF with a receptor antagonist may not be enough to arrest tumor growth. A number of angiogenesis inhibitors have been recently identified, and certain factors such as IFN-α, platelet-factor-4 (Maione, T. E. et al., 1990, Science 247:77-79) and PEX (Brooks, P. C. et al., 1998, Cell 92:391-400) are not endogenously associated with tumor cells, whereas angiostatin (O'Reilly, M. S. et al., 1994, Cell 79:315-328) and endostatin (O'Reilly, M. S. et al., 1997, Cell 88:277-285) are tumor associated angiogenesis inhibitors generated by tumor tissue itself. Although treatment of tumor growth and metastasis with these endogenous angiogenesis inhibitors is very effective and an attractive idea, some potential problems associated with anti-angiogenic therapies must be considered. Delayed toxicity induced by chronic anti-angiogenic therapy as well as the possibility of impaired wound healing and reproductive angiogenesis occurring during treatment are to be considered seriously.

In the present invention, a protein, and fragments, analogs, derivatives, homologs and mutants thereof with anti-angiogenic properties are described, along with methods of use of this protein, analogs, derivatives, homologs and mutants to inhibit angiogenesis-mediated proliferative diseases. The protein can comprise the G1 domain of the α1 chain of laminin, and is called “Matin.” This Matin protein is about 30 kDa, and inhibits endothelial cell proliferation.

Laminin is the most abundant noncollagenous protein found in basement membranes. It was initially purified from mouse EHS (Engelbreth-Holm-Swarm) tumors and the mouse embryonal carcinoma cell line M1536-B3. These oncogenic sources produce large amounts of easily extractable basement membrane-like substance, and most early research into components of the basement membrane used these tumor lines as sources, rather than naturally-occurring basement membranes. The patterns of gene expression are known to be different, however, between oncogenic and naturally- occurring tissues.

Laminin is a multidomain protein (Paulsson, M., 1992, Crit. Rev. Biochem. Mol. Biol. 27:93-127), with three distinct polypeptide chains, α, β1 and β2, connected into a cross shape by disulfide bonds. The N-terminal portion of each chain, containing domains III through VI, each forms one arm of the cross, and the C-terminal portions (containing domains I and II) of all three chains are joined together by disulfide bonds into the fourth arm. Put another way, the N-terminal half of the a chain makes up the vertical arms of the cross, while the N-terminal half of the β1 and β2 chains make up the left and right arms. The C-terminal halves of all three chains join together to form the lower vertical arm of the cross. The G domain only exists at the C-terminal end of the a chain, not on either of the β chains. The G domain is subdivided into five subdomains, G1 through G5. Merosin, an isoform of laminin, was found to share some amino acid identity with the C-terminus of the a chain of mouse laminin, and the general domain structure is conserved between the two.

Laminin and Matin can be obtained from a variety of sources. Such sources include, but are not limited to, P19137 (LAMININ ALPHA-1 CHAIN PRECURSOR (LAMININ A CHAIN)), MMMSA (laminin alpha-1 chain precursor—mouse) and AAA39410 (laminin A chain [Mus musculus]). Human laminin has a slightly lower sequence identity with SEQ ID NO: 2, e.g., around 83%. Such sequences are also useful for obtaining Matin, and include, but are not limited to, P25391 (LAMININ ALPHA-1 CHAIN PRECURSOR (LAMININ A CHAIN)), S14458 (laminin alpha-1 chain precursor—human) and CAA41418 (laminin A chain [Homo sapiens]). Other sequences have a lower identity with SEQ ID NO: 2, but may still be useful sources of anti-angiogenic Matin. These may include, but are not necessarily limited to, PX0082 (laminin, M chain—human (fragment)), MMHUMH (laminin alpha-2 chain—human (fragment)), AAB33989 (laminin M chain, merosin=basement membrane protein {G-domain} [human, placenta, Peptide Partial, 1751 aa] [Homo sapiens]), AAA63215 (merosin [Homo sapiens]), AAB18388 (laminin alpha 2 chain [Homo sapiens]), NP_{—}000417 (laminin alpha 2 subunit precursor; laminin, alpha-2 (merosin) [Homo sapiens]), P24043 (LAMININ ALPHA-2 CHAIN PRECURSOR (LAMININ M CHAIN) (MEROSIN HEAVY CHAIN)), CAA81394 (laminin M chain (merosin) [Homo sapiens]), XP_{—}011387 (laminin alpha 2 subunit precursor [Homo sapiens]), Q60675 (LAMININ ALPHA-2 CHAIN PRECURSOR (LAMININ M CHAIN) (MEROSIN HEAVY CHAIN)), S53868 (laminin alpha-2 chain precursor—mouse), AAC52165 (laminin-2 alpha2 chain precursor [Mus musculus]).

Interestingly, the various globular domains themselves possess varying levels of sequence identity with each other. This is shown in Table 1, below.

Table 1. Percent sequence identity of the globular domains of the mouse laminin α chain.

| | G1 | G2 | G3 | G4 | G5 |
|---|---|---|---|---|---|
| G1 | | | | | |
| G2 | 31 | | | | |
| G3 | — | 27 | | | |
| G4 | 24 | 25 | — | | |
| G5 | 23 | 31 | 27 | 25 | |

Polynucleotides encoding Matin can also be obtained from a variety of sources. For instance, other mouse laminin a chain globular domains (e.g., ) generally possess greater than 90% sequence identity with SEQ ID NO: 1, and include, but are not limited to, J04064 (Mus musculus laminin A chain mRNA, complete cds) and X58531 (Human LAMA mRNA for laminin A chain, partial cds).

As disclosed herein, laminin and/or Matin can be can be produced in E. coli using a bacterial expression plasmid, such as pET22b, which is capable of periplasmic transport, thus resulting in soluble protein. Laminin and/or Matin can also be produced in other cells, for instance, it can be produced as a secreted soluble protein in 293 kidney cells using the pcDNA 3.1 eukaryotic vector.

E. coli-produced Matin inhibits endothelial cell proliferation of endothelial cells in a dose-dependent manner.

Specific inhibition of endothelial cell proliferation and migration by Matin demonstrates its anti-angiogenic activity, and that it may function via a cell surface protein/receptor. Integrins are potential candidate molecules based on their extracellular matrix binding capacity and ability to modulate cell behavior such as migration and proliferation. In particular, aᵥb₃ integrin is a possible receptor, due to its induction during angiogenesis, and its promiscuous binding capacity. Angiogenesis also depends on specific endothelial cell adhesive events mediated by integrin aᵥb₃ (Brooks, P. C. et al., 1994, Cell 79:1157-1164). Matin may disrupt the interaction of proliferating endothelial cells to the matrix component, and thus drive endothelial cells to undergo apoptosis (Re, F. et al., 1994, J. Cell. Biol. 127:537-546). Matrix metalloproteinases (MMP's) have been implicated as key enzymes that regulate the formation of new blood vessels in tumors (Ray, J. M. et al., 1994, Eur. Respir. J. 7:2062-2072). Recently, it was demonstrated that an inhibitor of MMP-2 (PEX) can suppress tumor growth by inhibiting angiogenesis (Brooks, P. C. et al., 1998, Cell 92:391-400). Matin may function through inhibiting the activity of MMPs.

As used herein, the term “angiogenesis” means the generation of new blood vessels into a tissue or organ, and involves endothelial cell proliferation. Under normal physiological conditions, humans or animals undergo angiogenesis only in very specific restricted situations. For example, angiogenesis is normally observed in wound healing, fetal and embryonal development, and formation of the corpus luteum, endometrium and placenta. The term “endothelium” means a thin layer of flat epithelial cells that lines serous cavities, lymph vessels, and blood vessels. “Anti-angiogenic activity” therefore refers to the capability of a composition to inhibit the growth of blood vessels. The growth of blood vessels is a complex series of events, and includes localized breakdown of the basement membrane lying under the individual endothelial cells, proliferation of those cells, migration of the cells to the location of the future blood vessel, reorganization of the cells to form a new vessel membrane, cessation of endothelial cell proliferation, and, incorporation of pericytes and other cells that support the new blood vessel wall. “Anti-angiogenic activity” as used herein therefore includes interruption of any or all of these stages, with the end result that formation of new blood vessels is inhibited.

Anti-angiogenic activity may include endothelial inhibiting activity, which refers to the capability of a composition to inhibit angiogenesis in general and, for example, to inhibit the growth or migration of bovine capillary endothelial cells in culture in the presence of fibroblast growth factor, angiogenesis-associated factors, or other known growth factors. A “growth factor” is a composition that stimulates the growth, reproduction, or synthetic activity of cells. An “angiogenesis-associated factor” is a factor which either inhibits or promotes angiogenesis. An example of an angiogenesis-associated factor is an angiogenic growth factor, such as basic fibroblastic growth factor (bFGF), which is an angiogenesis promoter. Another example of an angiogenesis-associated factor is an angiogenesis inhibiting factor such as e.g., angiostatin (see, e.g., U.S. Pat. No. 5,801,012, U.S. Pat. No. 5,837,682, U.S. Pat. No. 5,733,876, U.S. Pat. No. 5,776,704, U.S. Pat. No. 5,639,725, U.S. Pat. No. 5,792,845, WO 96/35774, WO 95/29242, WO 96/41194, WO 97/23500) or endostatin (see, e.g., U.S. Pat. No. 5,854,205; U.S. Pat. No. 6,174,861; WO 97/15666).

By “substantially the same biological activity” or “substantially the same or superior biological activity” is meant that a composition has anti-angiogenic activity, and behaves similarly as does Matin, as determined in standard assays. “Standard assays” include, but are not limited to, those protocols used in the molecular biological arts to assess anti-angiogenic activity, cell cycle arrest, and apoptosis. Such assays include, but are not limited to, assays of endothelial cell proliferation, endothelial cell migration, cell cycle analysis, and endothelial cell tube formation, detection of apoptosis, e.g., by apoptotic cell morphology or Annexin V-FITC assay, chornoallantoic membrane (CAM) assay, and inhibition of renal cancer tumor growth in nude mice. Such assays are provided in the Examples below, and also in U.S. Ser. No. 09/335,224, “Anti-Angiogenic Proteins and Methods of Use thereof,” filed Jun. 17, 1999, by Raghuram Kalluri, and in U.S. Ser. No. 09/479,118, “Anti-Angiogenic Proteins and Receptors and Methods of Use thereof,” by Raghuram Kalluri, filed Jan. 7, 2000, all of which are incorporated herein by reference in their entirety.

“Laminin” is intended to include fragments, mutants, homologs, analogs, and allelic variants of laminin, as well as laminins from any mammal.

“Matin” is intended to include fragments, mutants, homologs, analogs, and allelic variants of the amino acid sequence of the Matin sequence, as well as Matin from other globular domains, globular domains from other ox chains, other laminins, laminins from other mammals, and fragments, mutants, homologs, analogs and allelic variants of the Matin amino acid sequence.

It is to be understood that the present invention is contemplated to include any derivatives of laminin or Matin that have endothelial inhibitory activity (e.g., the capability of a composition to inhibit angiogenesis in general and, for example, to inhibit the growth or migration of bovine capillary endothelial cells in culture in the presence of fibroblast growth factor, angiogenesis-associated factors, or other known growth factors). The present invention includes the entire Matin protein, derivatives of this protein and biologically-active fragments of this protein. This includes proteins with Matin activity that have amino acid substitutions or have sugars or other molecules attached to amino acid functional groups.

The invention also describes fragments, mutants, homologs and analogs of laminin and Matin. A “fragment” of a protein is defined herein as any amino acid sequence shorter than that protein, comprising at least 25 consecutive amino acids of the full polypeptide. Such a fragment may alternatively comprise 26, 27, 28, 29, 30, 31, 32, 33, 34, 35, 36, 37, 38, 39, 40, 41, 42, 43, 44, 45, 46, 47, 48, 49 or 50 consecutive amino acids of the full polypeptide. The fragment may comprise 51, 52, 53, 54, 55, 56, 57, 58, 59, 60, 61, 62, 63, 64, 65, 66, 67, 68, 69, 70, 71, 72, 73, 74 or 75 consecutive amino acids of the full polypeptide. Such molecules may or may not also comprise additional amino acids derived from the process of cloning, e.g., amino acid residues or amino acid sequences con-esponding to full or partial linker sequences. To be encompassed by the present invention, such molecules, with or without such additional amino acid residues, must have substantially the same biological activity as the reference polypeptide.

Where the full-length molecule possesses more than one activity, e.g., it may be possible to “split” the activities by splitting the full-length protein into several fragments, e.g., the full-length protein can be split into two fragments, one of which may possess one activity, while the other possesses another activity. The two fragments may or may not overlap, and the two activities may or may not be apparent in the full-length molecule. For instance, the full-length molecule may possess activity “A”, and two fragments whereof may possess activities “A₁” and “A₂”, respectively, or they may possess activities “B” and “C”. Therefore, when it is stated that a fragment or mutant “must have substantially the same biological activity as the reference polypeptide”, it is intended that in situations where one or more biological activities are split, the “reference polypeptide” is that subsequence of the overall molecule that corresponds to the fragment or mutant. That is, the fragment or mutant must have the substantially the same biological activity as that portion of the overall molecule to which they correspond.

By “mutant” of laminin or Matin is meant a polypeptide that includes any change in the amino acid sequence relative to the amino acid sequence of the equivalent reference laminin or Matin polypeptide repsectively. Such changes can arise either spontaneously or by manipulations by man, by chemical energy (e.g., X-ray), or by other forms of chemical mutagenesis, or by genetic engineering, or as a result of mating or other forms of exchange of genetic information. Mutations include, e.g., base changes, deletions, insertions, inversions, translocations, or duplications. Mutant forms of laminin or Matin may display either increased or decreased anti-angiogenic activity relative to the equivalent reference laminin or Matin polynucleotide respectively, and such mutants may or may not also comprise additional amino acids derived from the process of cloning, e.g., amino acid residues or amino acid sequences corresponding to full or partial linker sequences. Mutants/fragments of the anti-angiogenic proteins of the present invention can also be generated by PCR cloning, or by Pseudomonas elastase digestion, as described by Mariyama, M. et al. (1992, J. Biol. Chem. 267:1253-1258).

By “analog” of laminin or Matin is meant a non-natural molecule substantially similar to either the entire laminin or Matin molecule respectively, or a fragment or allelic variant thereof, and having substantially the same or superior biological activity. Such analogs are intended to include derivatives (e.g., chemical derivatives, as defined above) of the biologically active laminin or Matin, as well as its fragments, mutants, homologs, and allelic variants, which derivatives exhibit a qualitatively similar agonist or antagonist effect to that of the unmodified laminin or Matin polypeptide, fragment, mutant, homolog, or allelic variant respectively.

By “allele” of laminin or Matin is meant a polypeptide sequence containing a naturally-occurring sequence variation relative to the polypeptide sequence of the reference laminin or Matin polypeptide respectively. By “allele” of a polynucleotide encoding the laminin or Matin polypeptide is meant a polynucleotide containing a sequence variation relative to the reference polynucleotide sequence encoding the reference laminin or Matin polypeptide respectively, where the allele of the polynucleotide encoding the laminin or Matin polypeptide encodes an allelic form of the laminin or Matin polypeptide respectively.

It is possible that a given polypeptide may be either a fragment, a mutant, an analog, or allelic variant of laminin or Matin, or it may be two or more of those things, e.g., a polypeptide may be both an analog and a mutant of the laminin or Matin polypeptide respectively. For example, a shortened version of the Matin molecule (e.g., a fragment of Matin) may be created in the laboratory. If that fragment is then mutated through means known in the art, a molecule is created that is both a fragment and a mutant of Matin. In another example, a mutant may be created, which is later discovered to exist as an allelic form of Matin in some mammalian individuals. Such a mutant Matin molecule would therefore be both a mutant and an allelic variant. Such combinations of fragments, mutants, allelic variants, and analogs are intended to be encompassed in the present invention.

Encompassed by the present invention are proteins that have substantially the same amino acid sequence as laminin or Matin, or polynucleotides that have substantially the same nucleic acid sequence as the polynucleotides encoding laminin or Matin respectively. “Substantially the same sequence” means a nucleic acid or polypeptide that exhibits at least about 70% sequence identity with a reference sequence, e.g., another nucleic acid or polypeptide, typically at least about 80% sequence identity with the reference sequence, preferably at least about 90% sequence identity, more preferably at least about 95% identity, and most preferably at least about 97% sequence identity with the reference sequence. The length of comparison for sequences will generally be at least 75 nucleotide bases or 25 amino acids, more preferably at least 150 nucleotide bases or 50 amino acids, and most preferably 243-264 nucleotide bases or 81-88 amino acids. “Polypeptide” as used herein indicates a molecular chain of amino acids and does not refer to a specific length of the product. Thus, peptides, oligopeptides and proteins are included within the definition of polypeptide. This term is also intended to include polypeptide that have been subjected to post-expression modifications such as, for example, glycosylations, acetylations, phosphorylations and the like.

“Sequence identity,” as used herein, refers to the subunit sequence similarity between two polymeric molecules, e.g., two polynucleotides or two polypeptides. When a subunit position in both of the two molecules is occupied by the same monomeric subunit, e.g., if a position in each of two peptides is occupied by serine, then they are identical at that position. The identity between two sequences is a direct function of the number of matching or identical positions, e.g., if half (e.g., 5 positions in a polymer 10 subunits in length) of the positions in two peptide or compound sequences are identical, then the two sequences are 50% identical; if 90% of the positions, e.g., 9 of 10 are matched, the two sequences share 90% sequence identity. By way of example, the amino acid sequences R₂R₅R₇R₁₀R₆R₃ and R₉R₈R₁R₁₀R₆R₃ have 3 of 6 positions in common, and therefore share 50% sequence identity, while the sequences R₂R₅R₇R₁₀R₆R₃ and R₈R₁R₁₀R₆R₃ have 3 of 5 positions in common, and therefore share 60% sequence identity. The identity between two sequences is a direct function of the number of matching or identical positions. Thus, if a portion of the reference sequence is deleted in a particular peptide, that deleted section is not counted for purposes of calculating sequence identity, e.g., R₂R₅R₇R₁₀R₆R₃ and R₂R₅R₇R₁₀R₃ have 5 out of 6 positions in common, and therefore share 83.3% sequence identity.

Identity is often measured using sequence analysis software e.g., BLASTN or BLASTP. The default parameters for comparing two sequences (e.g., “Blast”-ing two sequences against each other by BLASTN (for nucleotide sequences) are reward for match=1, penalty for mismatch=−2, open gap=5, extension gap=2. When using BLASTP for protein sequences, the default parameters are reward for match=0, penalty for mismatch=0, open gap=11, and extension gap=1.

When two sequences share “sequence homology,” it is meant that the two sequences differ from each other only by conservative substitutions. For polypeptide sequences, such conservative substitutions consist of substitution of one amino acid at a given position in the sequence for another amino acid of the same class (e.g., amino acids that share characteristics of hydrophobicity, charge, pK or other conformational or chemical properties, e.g., valine for leucine, arginine for lysine), or by one or more non-conservative amino acid substitutions, deletions, or insertions, located at positions of the sequence that do not alter the conformation or folding of the polypeptide to the extent that the biological activity of the polypeptide is destroyed. Examples of “conservative substitutions” include substitution of one non-polar (hydrophobic) residue such as isoleucine, valine, leucine or methionine for one another; the substitution of one polar (hydrophilic) residue for another such as between arginine and lysine, between glutamine and asparagine, between threonine and serine; the substitution of one basic residue such as lysine, arginine or histidine for one another; or the substitution of one acidic residue, such as aspartic acid or glutamic acid for one another; or the use of a chemically derivatized residue in place of a non-derivatized residue; provided that the polypeptide displays the requisite biological activity. Two sequences which share sequence homology may called “sequence homologs.”

The invention contemplates mutants of the proteins and peptides disclosed herein, where the mutation(s) do not substantially alter the activity of the protein or peptide, that is the mutations are effectively “silent” mutations.

Homology, for polypeptides, is typically measured using sequence analysis software (e.g., Sequence Analysis Software Package of the Genetics Computer Group, University of Wisconsin Biotechnology Center, 1710 University Avenue, Madison, Wis. 53705). Protein analysis software matches similar sequences by assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Also encompassed by the present invention are chemical derivatives of laminin or Matin. “Chemical derivative” refers to a subject polypeptide having one or more residues chemically derivatized by reaction of a functional side group. Such derivatized residues include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-imbenzylhistidine. Also included as chemical derivatives are those peptides which contain one or more naturally occurring amino acid derivatives of the twenty standard amino acids. For examples: 4-hydroxyproline may be substituted for proline; 5-hydroxylysine may be substitute for lysine; 3-methylhistidine may be substituted for histidine; homoserine may be substituted for serine; and ornithine may be substituted for lysine.

The present invention also includes fusion proteins and chimeric proteins comprising the anti-angiogenic proteins, their fragments, mutants, homologs, analogs, and allelic variants. A fusion or chimeric protein can be produced as a result of recombinant expression and the cloning process, e.g., the protein may be produced comprising additional amino acids or amino acid sequences corresponding to full or partial linker sequences. A fusion or chimeric protein can consist of a multimer of a single protein, e.g., repeats of the anti-angiogenic proteins, or the fusion and chimeric proteins can be made up of several proteins, e.g., several of the anti-angiogenic proteins. The ftusion or chimeric protein can comprise a combination of two or more known anti-angiogenic proteins (e.g., angiostatin and endostatin, or biologically active fragments of angiostatin and endostatin), or an anti-angiogenic protein in combination with a targeting agent (e.g., endostatin with epidermal growth factor (EGF) or RGD peptides), or an anti-angiogenic protein in combination with an immunoglobulin molecule (e.g., endostatin and IgG, specifically with the Fc portion removed). The fusion and chimeric proteins can also include the anti-angiogenic proteins, their fragments, mutants, homologs, analogs, and allelic variants, and other anti-angiogenic proteins, e.g., endostatin, or angiostatin. Other anti-angiogenic proteins can include Arresten, Canstatin or Tumstatin (PCT/US99/13737, the entire teachings of which are herein incorporated by reference), Vascostatin, restin and apomigren (PCT/US98/26058, the entire teachings of which are herein incorporated by reference) and fragments of endostatin (PCT/US98/26057, the entire teachings of which are herein incorporated by reference). The term “fusion protein” or “chimeric protein” as used herein can also encompass additional components for e.g., delivering a chemotherapeutic agent, wherein a polynucleotide encoding the chemotherapeutic agent is linked to the polynucleotide encoding the anti-angiogenic protein. Fusion or chimeric proteins can also encompass multimers of an anti-angiogenic protein, e.g., a dimer or trimer. Such fusion or chimeric proteins can be linked together via post-translational modification (e.g., chemically linked), or the entire fusion protein may be made recombinantly.

Multimeric proteins comprising laminin or Matin, and fragments, mutants, homologs, analogs and allelic variants are also intended to be encompassed by the present invention. By “multimer” is meant a protein comprising two or more copies of a subunit protein. The subunit protein may be one of the proteins of the present invention, e.g., Matin repeated two or more times, or a fragment, mutant, homolog, analog or allelic variant, e.g., a Matin mutant or fragment, repeated two or more times. Such a multimer may also be a fusion or chimeric protein, e.g., a repeated tumstatin mutant may be combined with polylinker sequence, and/or one or more anti-angiogenic peptides, which may be present in a single copy, or may also be tandemly repeated, e.g., a protein may comprise two or more multimers within the overall protein.

The invention also encompasses a composition comprising one or more isolated polynucleotide(s) encoding laminin, as well as vectors and host cells containing such a polynucleotide, and processes for producing laminin and its fragments, mutants, homologs, analogs and allelic variants.

The invention also encompasses a composition comprising one or more isolated polynucleotide(s) encoding Matin, as well as vectors and host cells containing such a polynucleotide, and processes for producing Matin and its fragments, mutants, homologs, analogs and allelic variants. The term “vector” as used herein means a carrier into which pieces of nucleic acid may be inserted or cloned, which carrier functions to transfer the pieces of nucleic acid into a host cell. Such a vector may also bring about the replication and/or expression of the transferred nucleic acid pieces. Examples of vectors include nucleic acid molecules derived, e.g., from a plasmid, bacteriophage, or mammalian, plant or insect virus, or non-viral vectors such as ligand-nucleic acid conjugates, liposomes, or lipid-nucleic acid complexes. It may be desirable that the transferred nucleic molecule is operatively linked to an expression control sequence to form an expression vector capable of expressing the transferred nucleic acid. Such transfer of nucleic acids is generally called “transformation,” and refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome. “Operably linked” refers to a situation wherein the components described are in a relationship permitting them to function in their intended manner, e.g., a control sequence “operably linked” to a coding sequence is ligated in such a manner that expression of the coding sequence is achieved under conditions compatible with the control sequence. A “coding sequence” is a polynucleotide sequence which is transcribed into mRNA and translated into a polypeptide when placed under the control of (e.g., operably linked to) appropriate regulatory sequences. The boundaries of the coding sequence are determined by a translation start codon at the 5′-terminus and a translation stop codon at the 3′-terminus. Such boundaries can be naturally-occurring, or can be introduced into or added the polynucleotide sequence by methods known in the art. A coding sequence can include, but is not limited to, mRNA, cDNA, and recombinant polynucleotide sequences.

The vector into which the cloned polynucleotide is cloned may be chosen because it functions in a prokaryotic, or alternatively, it is chosen because it functions in a eukaryotic organism. Two examples of vectors which allow for both the cloning of a polynucleotide encoding the Matin protein, and the expression of that protein from the polynucleotide, are the pET22b and pET28(a) vectors (Novagen, Madison, Wis., USA) and a modified pPICZαA vector (InVitrogen, San Diego, Calif., USA), which allow expression of the protein in bacteria and yeast, respectively (see for example, WO 99/29878 and U.S. Ser. No. 09/589,483, the entire teachings which are hereby incorporated by reference).

Once a polynucleotide has been cloned into a suitable vector, it can be transformed into an appropriate host cell. By “host cell” is meant a cell which has been or can be used as the recipient of transferred nucleic acid by means of a vector. Host cells can prokaryotic or eukaryotic, mammalian, plant, or insect, and can exist as single cells, or as a collection, e.g., as a culture, or in a tissue culture, or in a tissue or an organism. Host cells can also be derived from normal or diseased tissue from a multicellular organism, e.g., a mammal. Host cell, as used herein, is intended to include not only the original cell which was transformed with a nucleic acid, but also descendants of such a cell, which still contain the nucleic acid.

In one embodiment, the isolated polynucleotide encoding the anti-angiogenic protein additionally comprises a polynucleotide linker encoding a peptide. Such linkers are known to those of skill in the art and, for example the linker can comprise at least one additional codon encoding at least one additional amino acid. Typically the linker comprises one to about twenty or thirty amino acids. The polynucleotide linker is translated, as is the polynucleotide encoding the anti-angiogenic protein, resulting in the expression of an anti-angiogenic protein with at least one additional amino acid residue at the amino or carboxyl terminus of the anti-angiogenic protein. Importantly, the additional amino acid, or amino acids, do not compromise the activity of the anti-angiogenic protein.

After inserting the selected polynucleotide into the vector, the vector is transformed into an appropriate prokaryotic strain and the strain is cultured (e.g., maintained) under suitable culture conditions for the production of the biologically active anti-angiogenic protein, thereby producing a biologically active anti-angiogenic protein, or mutant, derivative, fragment or fusion protein thereof. In one embodiment, the invention comprises cloning of a polynucleotide encoding an anti-angiogenic protein into the vectors pET22b, pET17b or pET28a, which are then transformed into bacteria. The bacterial host strain then expresses the anti-angiogenic protein. Typically the anti-angiogenic proteins are produced in quantities of about 10-20 milligrams, or more, per liter of culture fluid.

In another embodiment of the present invention, the eukaryotic vector comprises a modified yeast vector. One method is to use a pPICzα plasmid wherein the plasmid contains a multiple cloning site. The multiple cloning site has inserted into the multiple cloning site a His.Tag motif. Additionally the vector can be modified to add a NdeI site, or other suitable restriction sites. Such sites are well known to those of skill in the art. Anti-angiogenic proteins produced by this embodiment comprise a histidine tag motif (His.tag) comprising one, or more histidines, typically about 5-20 histidines. The tag must not interfere with the anti-angiogenic properties of the protein.

One method of producing Matin, for example, is to amplify the polynucleotide of SEQ ID NO: 1 and clone it into an expression vector, e.g., pET22b, pET28(a), pPICZαA, or some other expression vector, transform the vector containing the polynucleotide into a host cell capable of expressing the polypeptide encoded by the polynucleotide, culturing the transformed host cell under culture conditions suitable for expressing the protein, and then extracting and purifying the protein from the culture. Exemplary methods of producing anti-angiogenic proteins in general, are provided in the Examples below and in U.S. Ser. No. 09/335, 224, “Anti-Angiogenic Proteins and Methods of Use Thereof,” by Raghuram Kalluri, filed Jun. 17, 1999. The Matin protein may also be expressed as a product of transgenic animals, e.g., as a component of the milk of transgenic cows, goats, sheep or pigs, as is described in U.S. Pat. No. 5,962,648, or as a product of a transgenic plant, e.g., combined or linked with starch molecules in maize, or as is described in U.S. Pat. No. 5,639,947 or 5,990,385.

Matin may also be produced by conventional, known methods of chemical synthesis. Methods for constructing the proteins of the present invention by synthetic means are known to those skilled in the art. The synthetically-constructed Matin protein sequence, by virtue of sharing primary, secondary or tertiary structural and/or conformational characteristics with e.g., recombinantly-produced Matin, may possess biological properties in common therewith, including biological activity. Thus, the synthetically-constructed Matin protein sequence may be employed as biologically active or immunological substitutes for e.g., recombinantly-produced, purified Matin protein in screening of therapeutic compounds and in immunological processes for the development of antibodies.

The Matin protein is useful in inhibiting angiogenesis, as determined in standard assays, and provided in the Examples below.

Polynucleotides encoding laminin or Matin can be cloned out of isolated DNA or a cDNA library. Nucleic acids polypeptides, referred to herein as “isolated” are nucleic acids or polypeptides substantially free (i.e., separated away from) the material of the biological source from which they were obtained (e.g., as exists in a mixture of nucleic acids or in cells), which may have undergone further processing. “Isolated” nucleic acids or polypeptides include nucleic acids or polypeptides obtained by methods described herein, similar methods, or other suitable methods, including essentially pure nucleic acids or polypeptides, nucleic acids or polypeptides produced by chemical synthesis, by combinations of chemical or biological methods, and recombinantly produced nucleic acids or polypeptides which are isolated. An isolated polypeptide therefore means one which is relatively free of other proteins, carbohydrates, lipids, and other cellular components with which it is normally associated. An isolated nucleic acid is not immediately contiguous with (i.e., covalently linked to) both of the nucleic acids with which it is immediately contiguous in the naturally-occurring genome of the organism from which the nucleic acid is derived. The term, therefore, includes, for example, a nucleic acid which is incorporated into a vector (e.g., an autonomously replicating virus or plasmid), or a nucleic acid which exists as a separate molecule independent of other nucleic acids such as a nucleic acid fragment produced by chemical means or restriction endonuclease treatment.

The polynucleotides and proteins of the present invention can also be used to design probes to isolate other anti-angiogenic proteins. Exceptional methods are provided in U.S. Pat. No. 5,837,490, by Jacobs et al., the entire teachings of which are herein incorporated by reference in its entirety. The design of the oligonucleotide probe should preferably follow these parameters: (a) it should be designed to an area of the sequence which has the fewest ambiguous bases (“N's”), if any, and (b) it should be designed to have a Tₘ of approx. 80° C. (assuming 2° C. for each A or T and 4 degrees for each G or C).

The oligonucleotide should preferably be labeled with g-³²P-ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4×10⁶ dpm/pmole. The bacterial culture containing the pool of full-length clones should preferably be thawed and 100 μl of the stock used to inoculate a sterile culture flask containing 25 ml of sterile L-broth containing ampicillin at 100 μg/ml. The culture should preferably be grown to saturation at 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters and lyse, denature and bake them. Highly stringent condition are those that are at least as stringent as, for example, 1×SSC at 65° C., or 1×SSC and 50% formamide at 42° C. Moderate stringency conditions are those that are at least as stringent as 4×SSC at 65° C., or 4×SSC and 50% formnamide at 42° C. Reduced stringency conditions are those that are at least as stringent as 4×SSC at 50° C., or 6×SSC and 50% fonnamide at 40° C.

The filter is then preferably incubated at 65° C. for 1 hour with gentle agitation in 6×SSC (20×stock is 175.3 g NaCl/liter, 88.2 g Na citrate/liter, adjusted to pH 7.0 with NaOH) containing 0.5% SDS, 100 μg/ml of yeast RNA, and 10 mM EDTA (approximately 10 mL per 150 mm filter). Preferably, the probe is then added to the hybridization mix at a concentration greater than or equal to 1×10⁶ dpm/mL. The filter is then preferably incubated at 65° C. with gentle agitation overnight. The filter is then preferably washed in 500 mL of 2×SSC/0.5% SDS at room temperature without agitation, preferably followed by 500 mL of 2×SSC/0.1% SDS at room temperature with gentle shaking for 15 minutes. A third wash with 0.1×SSC/0.5% SDS at 65° C. for 30 minutes to 1 hour is optional. The filter is then preferably dried and subjected to autoradiography for sufficient time to visualize the positives on the X-ray film. Other known hybridization methods can also be employed. The positive colonies are then picked, grown in culture, and plasmid DNA isolated using standard procedures. The clones can then be verified by restriction analysis, hybridization analysis, or DNA sequencing.

Stringency conditions for hybridization refers to conditions of temperature and buffer composition which permit hybridization of a first nucleic acid sequence to a second nucleic acid sequence, wherein the conditions determine the degree of identity between those sequences which hybridize to each other. Therefore, “high stringency conditions” are those conditions wherein only nucleic acid sequences which are very similar to each other will hybridize. The sequences may be less similar to each other if they hybridize under moderate stringency conditions. Still less similarity is needed for two sequences to hybridize under low stringency conditions. By varying the hybridization conditions from a stringency level at which no hybridization occurs, to a level at which hybridization is first observed, conditions can be determined at which a given sequence will hybridize to those sequences that are most similar to it. The precise conditions determining the stringency of a particular hybridization include not only the ionic strength, temperature, and the concentration of destabilizing agents such as formamide, but also on factors such as the length of the nucleic acid sequences, their base composition, the percent of mismatched base pairs between the two sequences, and the frequency of occurrence of subsets of the sequences (e.g., small stretches of repeats) within other non-identical sequences. Washing is the step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature (Tₘ) for any chosen SSC concentration. Generally, a doubling of the concentration of SSC results in an increase in the Tₘ of about 17° C. Using these guidelines, the washing temperature can be determined empirically, depending on the level of mismatch sought. Hybridization and wash conditions are explained in Current Protocols in Molecular Biology (Ausubel, F. M. et al., eds., John Wiley & Sons, Inc., 1995, with supplemental updates) on pages 2.10.1 to 2.10.16, and 6.3.1 to 6.3.6.

High stringency conditions can employ hybridization at either (1) 1×SSC (10×SSC=3 M NaCl, 0.3 M Na₃-citrate·2H₂O (88 g/liter), pH to 7.0 with 1 M HCl), 1% SDS (sodium dodecyl sulfate), 0.1-2 mg/ml denatured salmon sperm DNA at 65° C., (2) 1×SSC, 50% formamide, 1% SDS, 0.1-2 mg/ml denatured salmon sperm DNA at 42° C., (3) 1% bovine serum albumen (fraction V), 1 mM Na₂·EDTA, 0.5 M NaHPO₄ (pH 7.2) (1 M NaHPO₄=134 g Na₂HPO₄·7H₂O, 4 ml 85% H₃PO₄ per liter), 7% SDS, 0.1-2 mg/ml denatured salmon sperm DNA at 65° C., (4) 50% formamide, 5×SSC, 0.02 M Tris-HCl (pH 7.6), 1×Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C., (5) 5×SSC, 5×Denhardt's solution, 1% SDS, 100 μg/ml denatured salmon sperm DNA at 65° C., or (6) 5×SSC, 5×Denhardt's solution, 50% formamide, 1% SDS, 100 μg/ml denatured salmon sperm DNA at 42° C., with high stringency washes of either (1) 0.3-0.1×SSC, 0.1% SDS at 65° C., or (2) 1 mM Na₂EDTA, 40 mM NaHPO₄ (pH 7.2), 1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2×the number of A and T bases)+(4×the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Moderate stringency conditions can employ hybridization at either (1) 4×SSC, (10×SSC=3 M NaCl, 0.3 M Na₃-citrate-2H₂O (88 g/liter), pH to 7.0 with 1 M HCl), 1% SDS (sodium dodecyl sulfate), 0.1-2 mg/ml denatured salmon sperm DNA at 65° C., (2) 4×SSC, 50% formamide, 1% SDS, 0.1-2 mg/ml denatured salmon spenn DNA at 42° C., (3) 1% bovine serum albumen (fraction V), 1 mM Na₂·EDTA, 0.5 M NaHPO₄ (pH 7.2) (1 M NaHPO₄=134 g Na₂HPO₄·7H₂O, 4 ml 85% H₃PO₄ per liter), 7% SDS, 0.1-2 mg/ml denatured salmon sperm DNA at 65° C., (4) 50% formamide, 5×SSC, 0.02 M Tris-HCl (pH 7.6), 1×Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C., (5) 5×SSC, 5×Denhardt's solution, 1% SDS, 100 μg/ml denatured salmon spenn DNA at 65° C., or (6) 5×SSC, 5×Denhardt's solution, 50% formamide, 1% SDS, 100 μg/ml denatured salmon sperm DNA at 42° C., with moderate stringency washes of 1×SSC, 0.1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2×the number of A and T bases) +(4×the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Low stringency conditions can employ hybridization at either (1) 4×SSC, (10×SSC=3 M NaCl, 0.3 M Na₃-citrate-2H₂O (88 g/liter), pH to 7.0 with 1 M HCl), 1% SDS (sodium dodecyl sulfate), 0.1-2 mg/ml denatured salmon sperm DNA at 50° C., (2) 6×SSC, 50% formamide, 1% SDS, 0.1-2 mg/ml denatured salmon sperm DNA at 40° C., (3) 1% bovine serum albumen (fraction V), 1 mM Na₂·EDTA, 0.5 M NaHPO₄ (pH 7.2) (1 M NaHPO₄=134 g Na₂HPO₄·7H₂O, 4 ml 85% H₃PO₄ per liter), 7% SDS, 0.1-2 mg/ml denatured salmon sperm DNA at 50° C., (4) 50% formamide, 5×SSC, 0.02 M Tris-HCl (pH 7.6), 1×Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 40° C., (5) 5×SSC, 5×Denhardt's solution, 1% SDS, 100 μg/ml denatured salmon sperm DNA at 50° C., or (6) 5×SSC, 5×Denhardt's solution, 50% formamide, 1% SDS, 100 μg/ml denatured salmon sperm DNA at 40° C., with low stringency washes of either 2×SSC, 0.1% SDS at 50° C., or (2) 0.5% bovine serum albumin (fraction V), 1 mM Na₂EDTA, 40 mM NaHPO₄ (pH 7.2), 5% SDS. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2×the number of A and T bases)+(4×the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

The present invention includes methods of inhibiting angiogenesis in mammalian tissue using laminin or its biologically-active fragments, analogs, homologs, derivatives or mutants.

The present invention includes methods of inhibiting angiogenesis in mammalian tissue using Matin or its biologically-active fragments, analogs, homologs, derivatives or mutants. In particular, the present invention includes methods of treating an angiogenesis-mediated disease with an effective amount of one or more of the anti-angiogenic proteins, or one or more biologically active fragment thereof, or combinations of fragments that possess anti-angiogenic activity, or agonists and antagonists. An effective amount of anti-angiogenic protein is an amount sufficient to inhibit the angiogenesis which results in the disease or condition, thus completely, or partially, alleviating the disease or condition. Alleviation of the angiogenesis-mediated disease can be determined by observing an alleviation of symptoms of the disease, e.g., a reduction in the size of a tumor, or arrested tumor growth. As used herein, the term “effective amount” also means the total amount of each active component of the composition or method that is sufficient to show a meaningful patient benefit, i.e., treatment, healing, prevention or amelioration of the relevant medical condition, or an increase in rate of treatment, healing, prevention or amelioration of such conditions. When applied to a combination, the term refers to combined amounts of the active ingredients that result in the therapeutic effect, whether administered in combination, serially or simultaneously. Angiogenesis-mediated diseases include, but are not limited to, cancers, solid tumors, blood-born tumors (e.g., leukemias), tumor metastasis, benign tumors (e.g., hemangiomas, acoustic neuromas, neurofibromas, organ fibrosis, trachomas, and pyogenic granulomas), rheumatoid arthritis, psoriasis, ocular angiogenic diseases (e.g., diabetic retinopathy, retinopathy of prematurity, macular degeneration, corneal graft rejection, neovascular glaucoma, retrolental fibroplasia, rubeosis), Osler-Webber Syndrome, myocardial angiogenesis, plaque neovascularization, telangiectasia, hemophiliac joints, angiofibroma, and wound granulation. The anti-angiogenic proteins are useful in the treatment of diseases of excessive or abnormal stimulation of endothelial cells. These diseases include, but are not limited to, intestinal adhesions, Crohn's disease, atherosclerosis, scleroderma, fibrosis and hypertrophic scars (i.e., keloids). The anti-angiogenic proteins can be used as a birth control agent by preventing vascularization required for embryo implantation. The anti-angiogenic proteins are useful in the treatment of diseases that have angiogenesis as a pathologic consequence such as cat scratch disease (Rochele minalia quintosa) and ulcers (Heliobacter pylori). The anti-angiogenic proteins can also be used to prevent dialysis graft vascular access stenosis, and obesity, e.g., by inhibiting capillary formation in adipose tissue, thereby preventing its expansion. The anti-angiogenic proteins can also be used to treat localized (e.g., nonmetastisized) diseases. “Cancer” means neoplastic growth, hyperplastic or proliferative growth or a pathological state of abnormal cellular development and includes solid tumors, non-solid tumors, and any abnormal cellular proliferation, such as that seen in leukemia. As used herein, “cancer” also means angiogenesis-dependent cancers and tumors, i.e., tumors that require for their growth (expansion in volume and/or mass) an increase in the number and density of the blood vessels supplying them with blood. “Regression” refers to the reduction of tumor mass and size as determined using methods well-known to those of skill in the art.

Alternatively, where an increase in angiogenesis is desired, e.g., in wound healing, or in post-infarct heart tissue, antibodies or antisera to the anti-angiogenic proteins can be used to block localized, native anti-angiogenic proteins and processes, and thereby increase formation of new blood vessels so as to inhibit atrophy of tissue.

The anti-angiogenic proteins may be used in combination with themselves, or other compositions and procedures for the treatment of diseases, e.g., Matin and Vascostatin can be combined in a pharmaceutical composition, one or more of their fragments can be combined in a composition, or a tumor may be treated conventionally with surgery, radiation, chemotherapy, or immunotherapy, combined with the anti-angiogenic proteins and then the anti-angiogenic proteins may be subsequently administered to the patient to extend the dormancy of micrometastases and to stabilize and inhibit the growth of any residual primary tumor. The anti-angiogenic proteins, or fragments, antisera, receptor agonists, or receptor antagonists thereof, or combinations thereof can also be combined with other anti-angiogenic compounds, or proteins, fragments, antisera, receptor agonists, receptor antagonists of other anti-angiogenic proteins (e.g., angiostatin, endostatin). Additionally, the anti-angiogenic proteins, or their fragments, antisera, receptor agonists, receptor antagonists, or combinations thereof, are combined with pharmaceutically acceptable excipients, and optionally sustained-release matrix, such as biodegradable polymers, to form therapeutic compositions. The compositions of the present invention mday also contain other anti-angiogenic proteins or chemical compounds, such as endostatin or angiostatin, and mutants, fragments, and analogs thereof. The compositions may further contain other agents which either enhance the activity of the protein or compliment its activity or use in treatment, such as chemotherapeutic or radioactive agents. Such additional factors and/or agents may be included in the composition to produce a synergistic effect with protein of the invention, or to minimize side effects. Additionally, administration of the composition of the present invention may be administered concurrently with other therapies, e.g., administered in conjunction with a chemotherapy or radiation therapy regimen.

The invention includes methods for inhibiting angiogenesis in mammalian (e.g., human) tissues by contacting the tissue with a composition comprising the proteins of the invention. By “contacting” is meant not only topical application, but also those modes of delivery that introduce the composition into the tissues, or into the cells of the tissues.

Use of timed release or sustained release delivery systems are also included in the invention. Such systems are highly desirable in situations where surgery is difficult or impossible, e.g., patients debilitated by age or the disease course itself, or where the risk-benefit analysis dictates control over cure.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The angiogenesis-modulating composition of the present invention may be a solid, liquid or aerosol and may be administered by any known route of administration. Examples of solid compositions include pills, creams, and implantable dosage units. The pills may be administered orally, the therapeutic creams may be administered topically. The implantable dosage unit may be administered locally, for example at a tumor site, or which may be implanted for systemic release of the angiogenesis-modulating composition, for example subcutaneously. Examples of liquid composition include formulations adapted for injection subcutaneously, intravenously, intraarterially, and formulations for topical and intraocular administration. Examples of aerosol formulation include inhaler formulation for administration to the lungs.

The proteins and protein fragments with the anti-angiogenic activity described above can be provided as isolated and substantially purified proteins and protein fragments in pharmaceutically acceptable formulations using formulation methods known to those of ordinary skill in the art. These formulations can be administered by standard routes. In general, the combinations may be administered by the topical, transdermal, intraperitoneal, intracranial, intracerebroventricular, intracerebral, intravaginal, intrauterine, oral, rectal or parenteral (e.g., intravenous, intraspinal, subcutaneous or intramuscular) route. In addition, the anti-angiogenic proteins may be incorporated into biodegradable polymers allowing for sustained release of the compound, the polymers being implanted in the vicinity of where drug delivery is desired, for example, at the site of a tumor or implanted so that the anti-angiogenic proteins are slowly released systemically. Osmotic minipumps may also be used to provide controlled delivery of high concentrations of the anti-angiogenic proteins through cannulae to the site of interest, such as directly into a metastatic growth or into the vascular supply to that tumor. The biodegradable polymers and their use are described, for example, in detail in Brem et al. (1991, J. Neurosurg. 74:441-6), which is hereby incorporated by reference in its entirety.

The compositions containing a polypeptide of this invention can be administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier or vehicle.

Modes of administration of the compositions of the present inventions include intravenous, intramuscular, intraperitoneal, intrasternal, subcutaneous and intraarticular injection and infusion. Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyois (e.g., glycerol, propylene glycol, polyethylene glycol and the like), carboxymethylcellulose and suitable mixtures thereof, vegetable oils (e.g., olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol sorbic acid and the like. It may also be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polmer employed, the rate of drug release can be controlled. Depot injectable formulations are also prepared by entrapping the drug in liposomes or microemulsions which are compatible with body tissues. The injectable formulations may be sterilized, for example, by filtration through a bacterial-retaining filter or by incorporating sterilizing agents in the form of sterile solid compositions which can be dissolved or dispersed in sterile water or other sterile injectable media just prior to use.

The therapeutic compositions of the present invention can include pharmaceutically acceptable salts of the components therein, e.g., which may be derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge, et al. describe pharmaceutically acceptable salts in detail in J. Pharmaceutical Sciences (1977) 66:1 et seq., which is incorporated herein by reference. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. The salts may be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptonoate, hexanoate, fumarate, hydrochloride, hydrobromide, hydroiodide, 2-hydroxymethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form phanmaceutically acceptable acid addition salts include such inorganic acids as hydrochloric acid, hydrobromic acid, sulphuric acid and phosphoric acid and such organic acids as oxalic acid, maleic acid, succinic acid and citric acid.

As used herein, the terms “phanmaceutically acceptable,” “physiologically tolerable” and grammatical variations thereof as they refer to compositions, carriers, diluents and reagents, are used interchangeably and represent that the materials are capable of administration to or upon a mammal with a minimum of undesirable physiological effects such as nausea, dizziness, gastric upset and the like. The preparation of a pharmacological composition that contains active ingredients dissolved or dispersed therein is well understood in the art and need not be limited based on fonnulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The anti-angiogenic proteins of the present invention can also be included in a composition comprising a prodrug. As used herein, the term “prodrug” refers to compounds which are rapidly transformed in vivo to yield the parent compound, for example, by enzymatic hydrolysis in blood. A thorough discussion is provided in T. Higuchi and V. Stella, Prodrugs as Novel Delivery Systems, Vol. 14 of the ACS Symposium Series and in Edward B. Roche, ed., Bioreversible Carriers in Drug Design, American Pharmaceutical Association and Permagon Press, 1987, both of which are incorporated herein by reference. As used herein, the term “pharmaceutically acceptable prodrug” refers to (1) those prodrugs of the compounds of the present invention which are, within the scope of sound medical judgement, suitable for use in contact with the tissues of humans and animals without undue toxicity, irritation, allergic response and the like, commensurate with a suitable benefit-to-risk ratio and effective for their intended use and (2) zwitterionic forms, where possible, of the parent compound.

The dosage of the anti-angiogenic proteins of the present invention will depend on the disease state or condition being treated and other clinical factors such as weight and condition of the human or animal and the route of administration of the compound. Depending upon the half-life of the anti-angiogenic proteins in the particular animal or human, the anti-angiogenic proteins can be administered between several times per day to once a week. It is to be understood that the present invention has application for both human and veterinary use. The methods of the present invention contemplate single as well as multiple administrations, given either simultaneously or over an extended period of time. In addition, the anti-angiogenic proteins can be administered in conjunction with other forms of therapy, e.g., chemotherapy, radiotherapy, or immunotherapy.

The anti-angiogenic protein formulations include those suitable for oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) administration. The anti-angiogenic protein formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

When an effective amount of protein of the present invention is administered orally, the anti-angiogenic proteins of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

When an effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of protein of the present invention in the pharmaceutical composition of the present invention will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Preferred unit dosage formnulations are those containing a daily dose or unit, daily sub-dose, or an appropriate fraction thereof, of the administered ingredient. It should be understood that in addition to the ingredients, particularly mentioned above, the formulations of the present invention may include other agents conventional in the art having regard to the type of formulation in question. Optionally, cytotoxic agents may be incorporated or otherwise combined with the anti-angiogenic proteins, or biologically functional protein fragements thereof, to provide dual therapy to the patient.

The therapeutic compositions are also presently valuable for veterinary applications. Particularly domestic animals and thoroughbred horses, in addition to humans, are desired patients for such treatment with proteins of the present invention.

Cytotoxic agents such as ricin, can be linked to the anti-angiogenic proteins, and fragments thereof, thereby providing a tool for destruction of cells that bind the anti-angiogenic proteins. These cells may be found in many locations, including but not limited to, micrometastases and primary tumors. Proteins linked to cytotoxic agents are infused in a manner designed to maximize delivery to the desired location. For example, ricin-linked high affinity fragments are delivered through a cannula into vessels supplying the target site or directly into the target. Such agents are also delivered in a controlled manner through osmotic pumps coupled to infusion cannulac. A combination of antagonists to the anti-angiogenic proteins may be co-applied with stimulators of angiogenesis to increase vascularization of tissue. This therapeutic regimen provides an effective means of destroying metastatic cancer.

Additional treatment methods include administration of the anti-angiogenic proteins, fragments, analogs, antisera, or receptor agonists and antagonists thereof, linked to cytotoxic agents. It is to be understood that the anti-angiogenic proteins can be human or animal in origin. The anti-angiogenic proteins can also be produced synthetically by chemical reaction or by recombinant techniques in conjunction with expression systems. The anti-angiogenic proteins can also be produced by enzymatically cleaving isolated laminin to generate proteins having anti-angiogenic activity. The anti-angiogenic proteins may also be produced by compounds that mimic the action of endogenous enzymes that cleave laminin to the anti-angiogenic proteins. Production of the anti-angiogenic proteins may also be modulated by compounds that affect the activity of cleavage enzymes.

The present invention also encompasses gene therapy whereby a polynucleotide encoding the anti-angiogenic proteins, integrins, integrin subunits, or a mutant, fragment, or fusion protein thereof, is introduced and regulated in a patient. Various methods of transferring or delivering DNA to cells for expression of the gene product protein, otherwise referred to as gene therapy, are disclosed in Gene Transfer into Mammalian Somatic Cells in vivo, N. Yang (1992) Crit. Rev. Biotechn. 12(4):335-56, which is hereby incorporated by reference. Gene therapy encompasses incorporation of DNA sequences into somatic cells or germ line cells for use in either ex vivo or in vivo therapy. Gene therapy functions to replace genes, augment normal or abnormal gene function, and to combat infectious diseases and other pathologies.

Strategies for treating these medical problems with gene therapy include therapeutic strategies such as identifying the defective gene and then adding a functional gene to either replace the function of the defective gene or to augment a slightly functional gene; or prophylactic strategies, such as adding a gene for the product protein that will treat the condition or that will make the tissue or organ more susceptible to a treatment regimen. As an example of a prophylactic strategy, a gene such as that encoding one or more of the anti-angiogenic proteins may be placed in a patient and thus prevent occurrence of angiogenesis; or a gene that makes tumor cells more susceptible to radiation could be inserted and then radiation of the tumor would cause increased killing of the tumor cells.

Many protocols for transfer of the DNA or regulatory sequences of the anti-angiogenic proteins are envisioned in this invention. Transfection of promoter sequences, other than one normally found specifically associated with the anti-angiogenic proteins, or other sequences which would increase production of the anti-angiogenic proteins are also envisioned as methods of gene therapy. An example of this technology is found in Transkaryotic Therapies, Inc., of Cambridge, Mass., using homologous recombination to insert a “genetic switch” that turns on an erythropoietin gene in cells. See Genetic Engineering News, Apr. 15, 1994. Such “genetic switches” could be used to activate the anti-angiogenic proteins (or their receptors) in cells not normally expressing those proteins (or receptors).

Gene transfer methods for gene therapy fall into three broad categories: physical (e.g., electroporation, direct gene transfer and particle bombardment), chemical (e.g., lipid-based carriers, or other non-viral vectors) and biological (e.g., virus-derived vector and receptor uptake). For example, non-viral vectors may be used which include liposomes coated with DNA. Such liposome/DNA complexes may be directly injected intravenously into the patient. It is believed that the liposome/DNA complexes are concentrated in the liver where they deliver the DNA to macrophages and Kupffer cells. These cells are long lived and thus provide long term expression of the delivered DNA. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, the transfected cells are expanded in number and then reimplanted in the patient. In in vitro gene transfer, the transformed cells are cells growing in culture, such as tissue culture cells, and not particular cells from a particular patient. These “laboratory cells” are transfected, the transfected cells are selected and expanded for either implantation into a patient or for other uses.

In vivo gene transfer involves introducing the DNA into the cells of the patient when the cells are within the patient. Methods include using virally mediated gene transfer using a noninfectious virus to deliver the gene in the patient or injecting naked DNA into a site in the patient and the DNA is taken up by a percentage of cells in which the gene product protein is expressed. Additionally, the other methods described herein, such as use of a “gene gun,” may be used for in vitro insertion of the DNA or regulatory sequences controlling production of the anti-angiogenic proteins.

Chemical methods of gene therapy may involve a lipid based compound, not necessarily a liposome, to transfer the DNA across the cell membrane. Lipofectins or cytofectins, lipid-based positive ions that bind to negatively charged DNA, make a complex that can cross the cell membrane and provide the DNA into the interior of the cell. Another chemical method uses receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then target cells that have the receptor will specifically bind the ligand and transport the ligand-DNA complex into the cell.

Many gene therapy methodologies employ viral vectors to insert genes into cells. For example, altered retrovirus vectors have been used in ex vivo methods to introduce genes into peripheral and tumor-infiltrating lymphocytes, hepatocytes, epidermal cells, myocytes, or other somatic cells. These altered cells are then introduced into the patient to provide the gene product from the inserted DNA.

Viral vectors have also been used to insert genes into cells using in vivo protocols. To direct the tissue-specific expression of foreign genes, cis-acting regulatory elements or promoters that are known to be tissue-specific can be used. Alternatively, this can be achieved using in situ delivery of DNA or viral vectors to specific anatomical sites in vivo. For example, gene transfer to blood vessels in vivo was achieved by implanting in vitro transduced endothelial cells in chosen sites on arterial walls. The virus infected surrounding cells which also expressed the gene product. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors that have been used for gene therapy protocols include but are not limited to, retroviruses, other RNA viruses such as poliovirus or Sindbis virus, adenovirus, adeno-associated virus, herpes viruses, SV 40, vaccinia and other DNA viruses. Replication-defective murine retroviral vectors are the most widely utilized gene transfer vectors. Murine leukemia retroviruses are composed of a single strand RNA complexed with a nuclear core protein and polymerase (pol) enzymes, encased by a protein core (gag) and surrounded by a glycoprotein envelope (env) that determines host range. The genomic structure of retroviruses include the gag, pol, and env genes enclosed at by the 5′ and 3′ long terminal repeats (LTR). Retroviral vector systems exploit the fact that a minimal vector containing the 5′ and 3′ LTRs and the packaging signal are sufficient to allow vector packaging, infection and integration into target cells providing that the viral structural proteins are supplied in trans in the packaging cell line. Fundamental advantages of retroviral vectors for gene transfer include efficient infection and gene expression in most cell types, precise single copy vector integration into target cell chromosomal DNA, and ease of manipulation of the retroviral genome.

The adenovirus is composed of linear, double stranded DNA complexed with core proteins and surrounded with capsid proteins. Advances in molecular virology have led to the ability to exploit the biology of these organisms to create vectors capable of transducing novel genetic sequences into target cells in vivo. Adenoviral-based vectors will express gene product proteins at high levels. Adenoviral vectors have high efficiencies of infectivity, even with low titers of virus. Additionally, the virus is fully infective as a cell free virion so injection of producer cell lines is not necessary. Another potential advantage to adenoviral vectors is the ability to achieve long term expression of heterologous genes in vivo.

Mechanical methods of DNA delivery include fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun,” and inorganic chemical approaches such as calcium phosphate transfection. Particle-mediated gene transfer methods were first used in transforming plant tissue. With a particle bombardment device, or “gene gun,” a motive force is generated to accelerate DNA-coated high density particles (such as gold or tungsten) to a high velocity that allows penetration of the target organs, tissues or cells. Particle bombardment can be used in in vitro systems, or with ex vivo or in vivo techniques to introduce DNA into cells, tissues or organs. Another method, ligand-mediated gene therapy, involves complexing the DNA with specific ligands to form ligand-DNA conjugates, to direct the DNA to a specific cell or tissue.

It has been found that injecting plasmid DNA into muscle cells yields high percentage of the cells which are transfected and have sustained expression of marker genes. The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Electroporation for gene transfer uses an electrical current to make cells or tissues susceptible to electroporation-mediated mediated gene transfer. A brief electric impulse with a given field strength is used to increase the permeability of a membrane in such a way that DNA molecules can penetrate into the cells. This technique can be used in in vitro systems, or with ex vivo or in vivo techniques to introduce DNA into cells, tissues or organs.

Carrier mediated gene transfer in vivo can be used to transfect foreign DNA into cells. The carrier-DNA complex can be conveniently introduced into body fluids or the bloodstream and then site-specifically directed to the target organ or tissue in the body. Both liposomes and polycations, such as polylysine, lipofectins or cytofectins, can be used. Liposomes can be developed which are cell specific or organ specific and thus the foreign DNA carried by the liposome will be taken up by target cells. Injection of immunoliposomes that are targeted to a specific receptor on certain cells can be used as a convenient method of inserting the DNA into the cells bearing the receptor. Another carrier system that has been used is the asialoglycoportein/polylysine conjugate system for carrying DNA to hepatocytes for in vivo gene transfer.

The transfected DNA may also be complexed with other kinds of carriers so that the DNA is carried to the recipient cell and then resides in the cytoplasm or in the nucleoplasm. DNA can be coupled to carrier nuclear proteins in specifically engineered vesicle complexes and carried directly into the nucleus.

Gene regulation of the anti-angiogenic proteins may be accomplished by administering compounds that bind to the gene encoding one of the anti-angiogenic proteins, or control regions associated with the gene, or its corresponding RNA transcript to modify the rate of transcription or translation. Additionally, cells transfected with a DNA sequence encoding the anti-angiogenic proteins may be administered to a patient to provide an in vivo source of those proteins. For example, cells may be transfected with a vector containing a nucleic acid sequence encoding the anti-angiogenic proteins. The transfected cells may be cells derived from the patient's normal tissue, the patient's diseased tissue, or may be non-patient cells.

For example, tumor cells removed from a patient can be transfected with a vector capable of expressing the proteins of the present invention, and re-introduced into the patient. The transfected tumor cells produce levels of the protein in the patient that inhibit the growth of the tumor. Patients may be human or non-human animals. Cells may also be transfected by non-vector, or physical or chemical methods known in the art such as electroporation, ionoporation, or via a “gene gun.” Additionally, the DNA may be directly injected, without the aid of a carrier, into a patient. In particular, the DNA may be injected into skin, muscle or blood.

The gene therapy protocol for transfecting the anti-angiogenic proteins into a patient may either be through integration of the anti-angiogenic protein DNA into the genome of the cells, into minichromosomes or as a separate replicating or non-replicating DNA construct in the cytoplasm or nucleoplasm of the cell. Expression of the anti-angiogenic proteins may continue for a long-period of time or may be reinjected periodically to maintain a desired level of the protein(s) in the cell, the tissue or organ or a determined blood level.

In addition, the invention encompasses antibodies and antisera, which can be used for testing of novel anti-angiogenic proteins, and can also be used in diagnosis, prognosis, or treatment of diseases and conditions characterized by, or associated with, angiogenic activity or lack thereof. Such antibodies and antisera can also be used to up-regulate angiogenesis where desired, e.g., in post-infarct heart tissue, antibodies or antisera to the proteins of the invention can be used to block localized, native anti-angiogenic proteins and processes, and increase formation of new blood vessels and inhibit atrophy of heart tissue.

Such antibodies and antisera can be combined with pharmaceutically-acceptable compositions and carriers to form diagnostic, prognostic or therapeutic compositions. The term “antibody” or “antibody molecule” refers to a population of immunoglobulin molecules and/or immunologically active portions of immunoglobulin molecules, i.e., molecules that contain an antibody combining site or paratope.

Passive antibody therapy using antibodies that specifically bind the anti-angiogenic proteins can be employed to modulate angiogenic-dependent processes such as reproduction, development, and wound healing and tissue repair. In addition, antisera directed to the Fab regions of antibodies of the anti-angiogenic proteins can be administered to block the ability of endogenous antisera to the proteins to bind the proteins.

The the anti-angiogenic proteins of the present invention also can be used to generate antibodies that are specific for the inhibitor(s) and receptor(s). The antibodies can be either polyclonal antibodies or monoclonal antibodies. These antibodies that specifically bind to the anti-angiogenic proteins or their receptors can be used in diagnostic methods and kits that are well known to those of ordinary skill in the art to detect or quantify the anti-angiogenic proteins or their receptors in a body fluid or tissue. Results from these tests can be used to diagnose or predict the occurrence or recurrence of a cancer and other angiogenic mediated diseases.

The invention also includes use of the anti-angiogenic proteins, antibodies to those proteins, and compositions comprising those proteins and/or their antibodies in diagnosis or prognosis of diseases characterized by angiogenic activity. As used herein, the term “prognostic method” means a method that enables a prediction regarding the progression of a disease of a human or animal diagnosed with the disease, in particular, an angiogenesis dependent disease. The term “diagnostic method” as used herein means a method that enables a determination of the presence or type of angiogenesis-dependent disease in or on a human or animal.

The the anti-angiogenic proteins can be used in a diagnostic method and kit to detect and quantify antibodies capable of binding the proteins. These kits would permit detection of circulating antibodies to the anti-angiogenic proteins which indicates the spread of micrometastases in the presence of the anti-angiogenic proteins secreted by primary tumors in situ. Patients that have such circulating anti-protein antibodies may be more likely to develop multiple tumors and cancers, and may be more likely to have recurrences of cancer after treatments or periods of remission. The Fab fragments of these anti-protein antibodies may be used as antigens to generate anti-protein Fab-fragment antisera which can be used to neutralize anti-protein antibodies. Such a method would reduce the removal of circulating protein by anti-protein antibodies, thereby effectively elevating circulating levels of the anti-angiogenic proteins.

The present invention also includes isolation of receptors specific for the anti-angiogenic proteins. Protein fragments that possess high affinity binding to tissues can be used to isolate the receptor of the anti-angiogenic proteins on affinity columns. Isolation and purification of the receptor(s) is a fundamental step towards elucidating the mechanism of action of the anti-angiogenic proteins. Isolation of a receptor and identification of agonists and antagonists will facilitate development of drugs to modulate the activity of the receptor, the final pathway to biological activity. Isolation of the receptor enables the construction of nucleotide probes to monitor the location and synthesis of the receptor, using in situ and solution hybridization technology. Further, the gene for the receptor can be isolated, incorporated into an expression vector and transfected into cells, such as patient tumor cells to increase the ability of a cell type, tissue or tumor to bind the anti-angiogenic proteins and inhibit local angiogenesis.

The anti-angiogenic proteins are employed to develop affinity columns for isolation of the receptor(s) for the anti-angiogenic proteins from cultured tumor cells. Isolation and purification of the receptor is followed by amino acid sequencing. Using this information the gene or genes coding for the receptor can be identified and isolated. Next, cloned nucleic acid sequences are developed for insertion into vectors capable of expressing the receptor. These techniques are well known to those skilled in the art. Transfection of the nucleic acid sequence(s) coding for the receptor into tumor cells, and expression of the receptor by the transfected tumor cells enhances the responsiveness of these cells to endogenous or exogenous anti-angiogenic proteins and thereby decreasing the rate of metastatic growth.

Angiogenesis-inhibiting proteins of the present invention can be synthesized in a standard microchemical facility and purity checked with HPLC and mass spectrophotometry. Methods of protein synthesis, HPLC purification and mass spectrophotometry are commonly known to those skilled in these arts. The anti-angiogenic proteins and their receptors proteins are also produced in recombinant E. coli or yeast expression systems, and purified with column chromatography.

Different protein fragments of the intact the anti-angiogenic proteins can be synthesized for use in several applications including, but not limited to the following; as antigens for the development of specific antisera, as agonists and antagonists active at binding sites of the anti-angiogenic proteins, as proteins to be linked to, or used in combination with, cytotoxic agents for targeted killing of cells that bind the anti-angiogenic proteins.

The synthetic protein fragments of the anti-angiogenic proteins have a variety of uses. The protein that binds to the receptor(s) of the anti-angiogenic proteins with high specificity and avidity is radiolabeled and employed for visualization and quantitation of binding sites using autoradiographic and membrane binding techniques. This application provides important diagnostic and research tools. Knowledge of the binding properties of the receptor(s) facilitates investigation of the transduction mechanisms linked to the receptor(s).

The anti-angiogenic proteins and proteins derived from them can be coupled to other molecules using standard methods. The amino and carboxyl termini of the anti-angiogenic proteins both contain tyrosine and lysine residues and are isotopically and nonisotopically labeled with many techniques, for example radiolabeling using conventional techniques (tyrosine residues-chloramine T, iodogen, lactoperoxidase; lysine residues-Bolton-Hunter reagent). These coupling techniques are well known to those skilled in the art. Alternatively, tyrosine or lysine is added to fragments that do not have these residues to facilitate labeling of reactive amino and hydroxyl groups on the protein. The coupling technique is chosen on the basis of the functional groups available on the amino acids including, but not limited to amino, sulfhydral, carboxyl, amide, phenol, and imidazole. Various reagents used to effect these couplings include among others, glutaraldehyde, diazotized benzidine, carbodiimide, and p-benzoquinone.

The anti-angiogenic proteins are chemically coupled to isotopes, enzymes, carrier proteins, cytotoxic agents, fluorescent molecules, chemiluminescent, bioluminescent and other compounds for a variety of applications. The efficiency of the coupling reaction is determined using different techniques appropriate for the specific reaction. For example, radiolabeling of a protein of the present invention with ¹²⁵I is accomplished using chloramine T and Na ¹²⁵I of high specific activity. The reaction is terminated with sodium metabisulfite and the mixture is desalted on disposable columns. The labeled protein is eluted from the column and fractions are collected. Aliquots are removed from each fraction and radioactivity measured in a gamma counter. In this manner, the unreacted Na ¹²⁵I is separated from the labeled protein. The protein fractions with the highest specific radioactivity are stored for subsequent use such as analysis of the ability to bind to antisera of the anti-angiogenic proteins.

In addition, labeling the anti-angiogenic proteins with short lived isotopes enables visualization of receptor binding sites in vivo using positron emission tomography or other modern radiographic techniques to locate tumors with the proteins' binding sites.

Systematic substitution of amino acids within these synthesized proteins yields high affinity protein agonists and antagonists to the receptor(s) of the anti-angiogenic proteins that enhance or diminish binding to the receptor(s). Such agonists are used to suppress the growth of micrometastases, thereby limiting the spread of cancer. Antagonists to the anti-angiogenic proteins are applied in situations of inadequate vascularization, to block the inhibitory effects of the anti-angiogenic proteins and promote angiogenesis. For example, this treatment may have therapeutic effects to promote wound healing in diabetics.

The invention is further illustrated by the following examples, which are not meant to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Example 1

### Recombinant Production of Matin in E. coli.

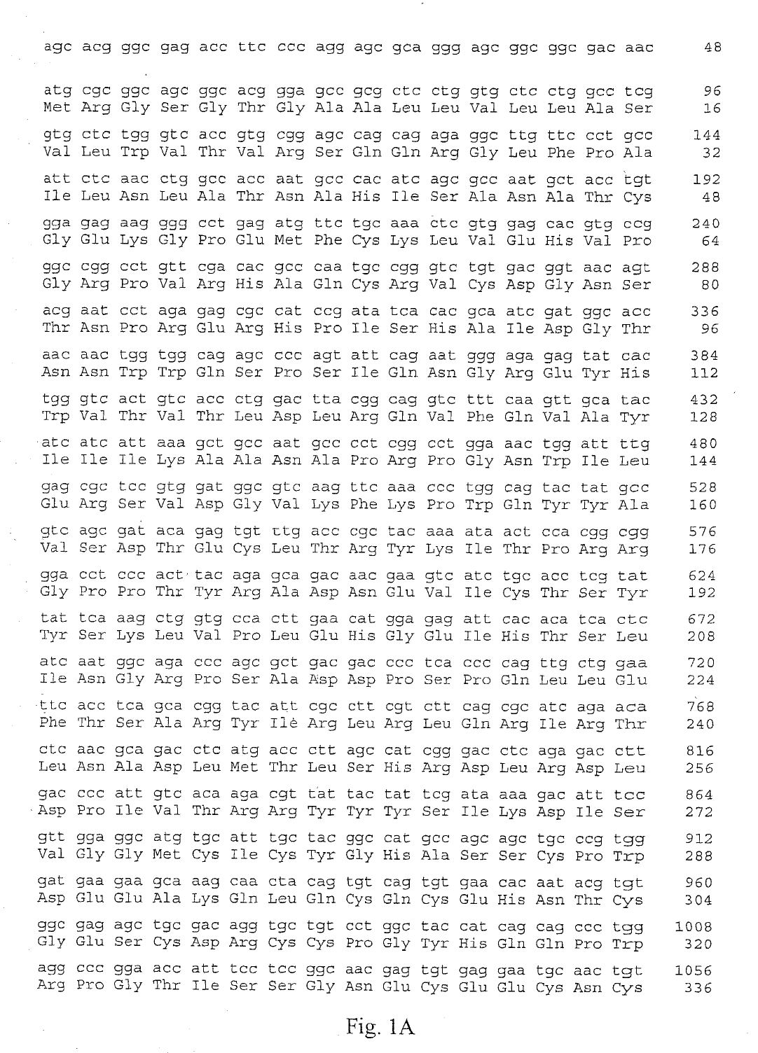

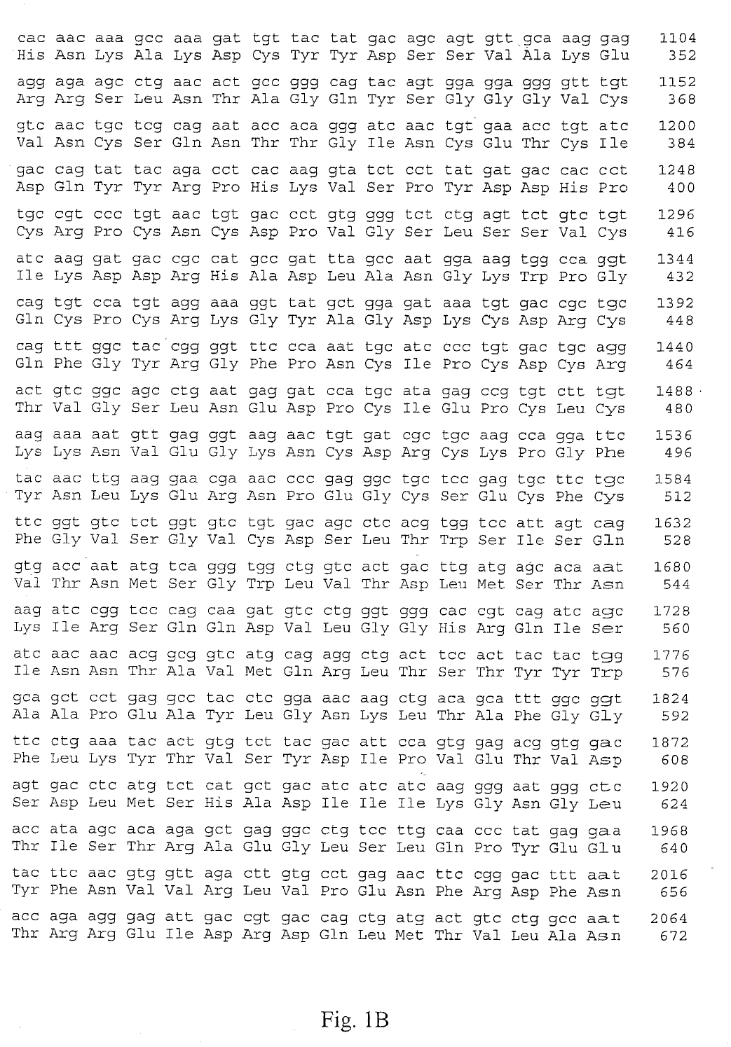

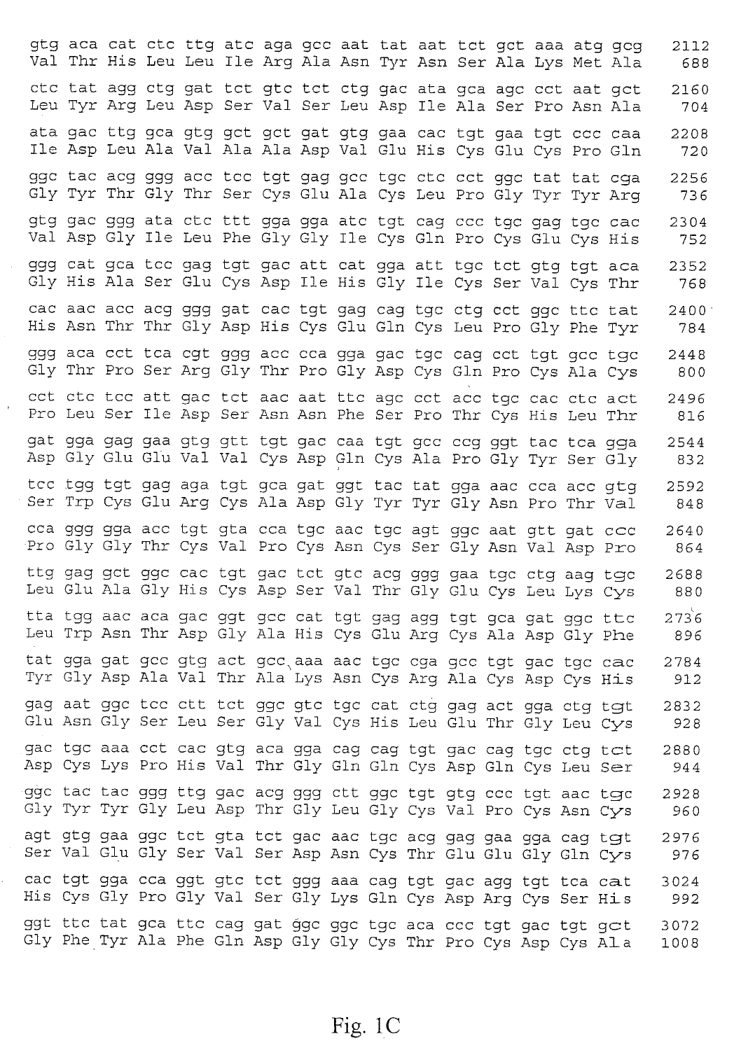

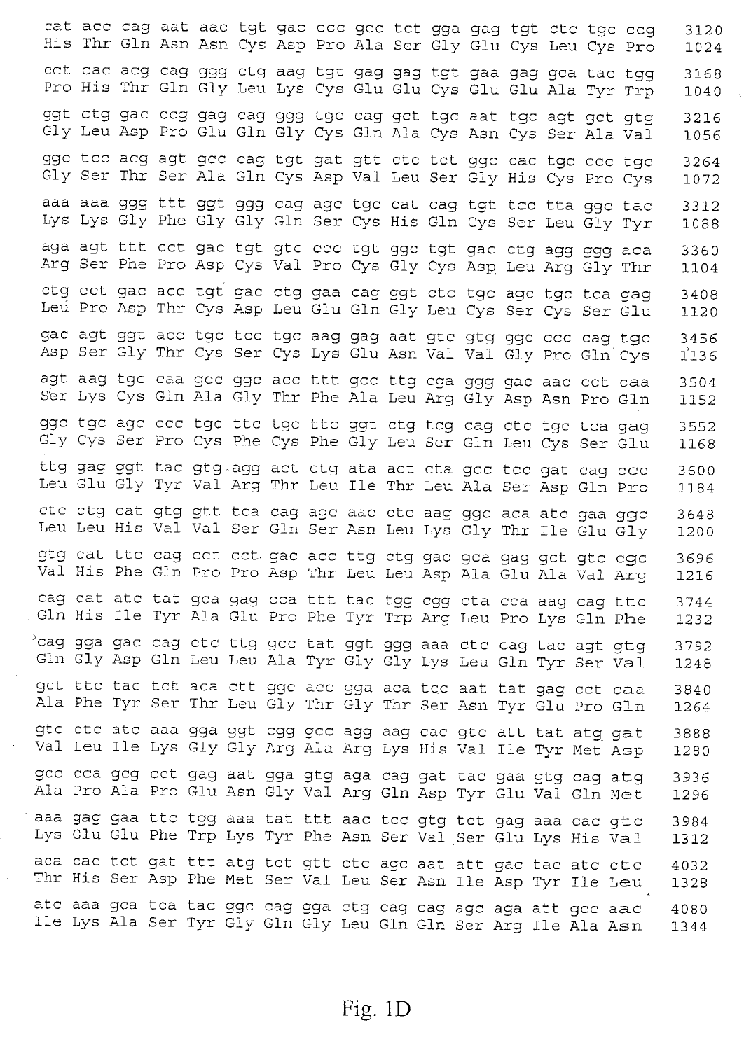

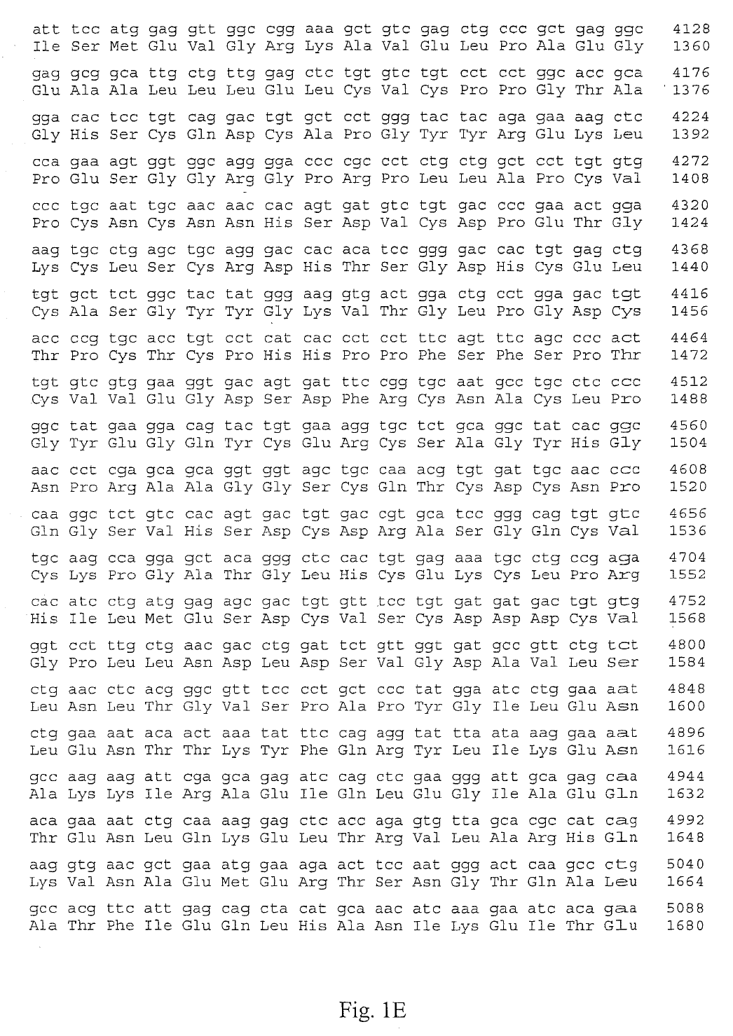

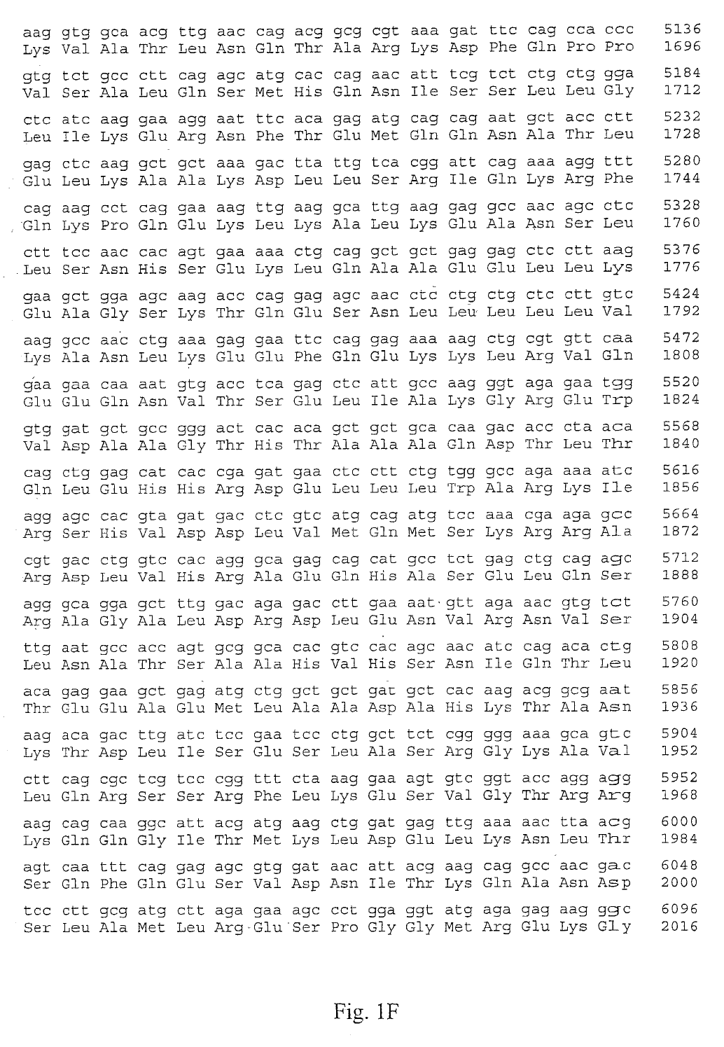

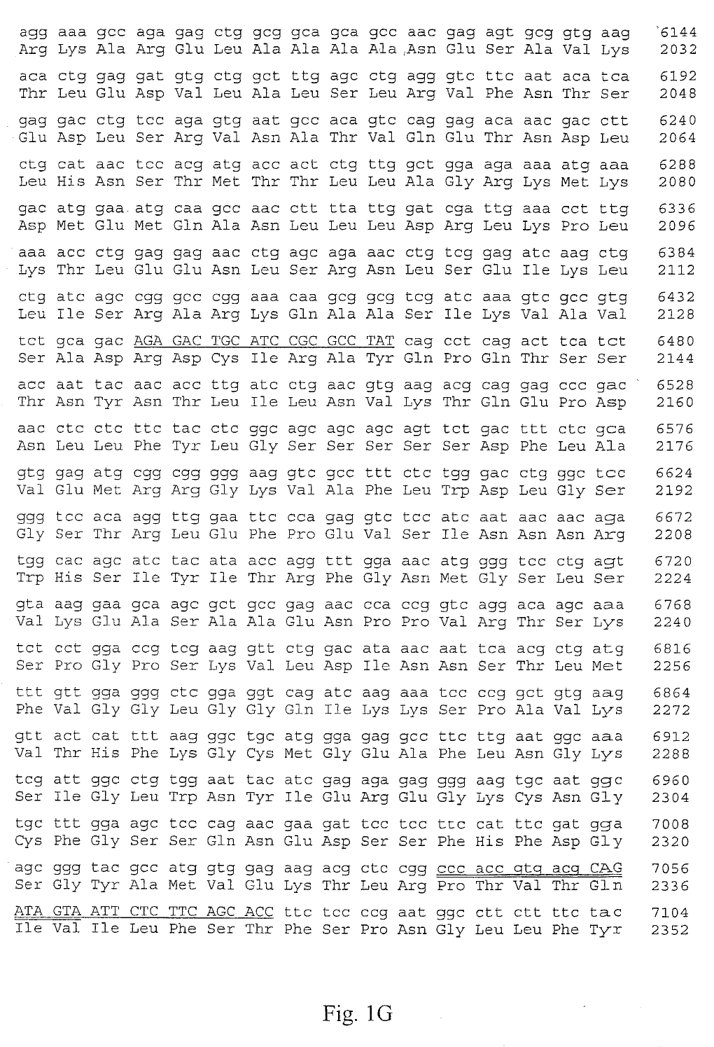

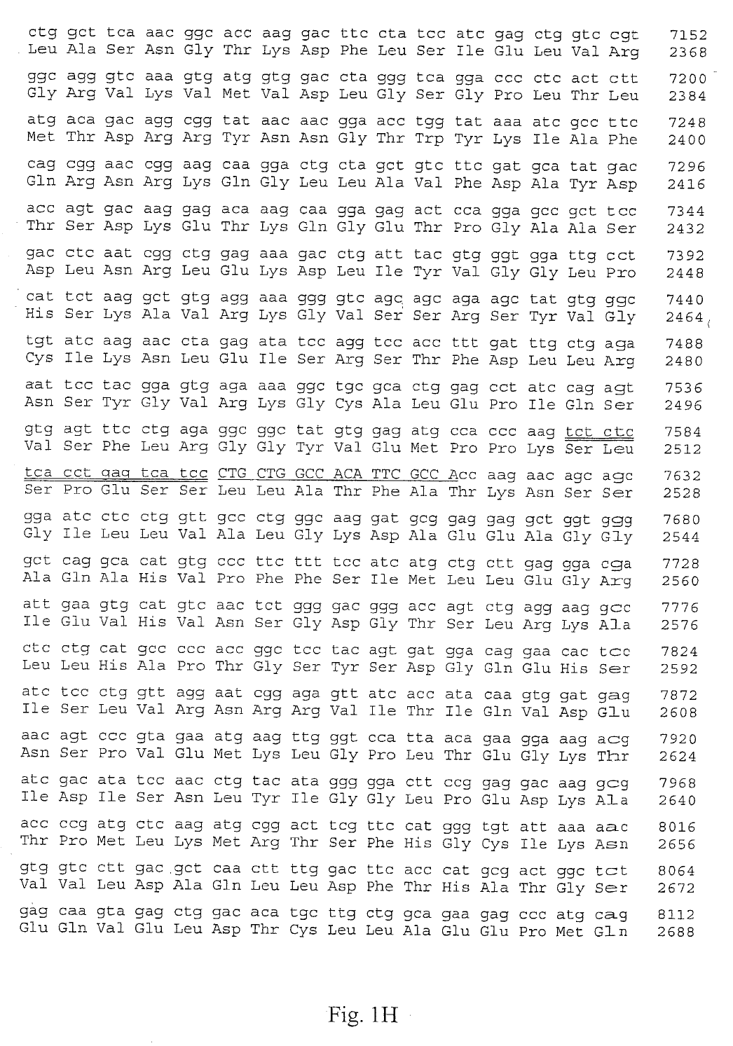

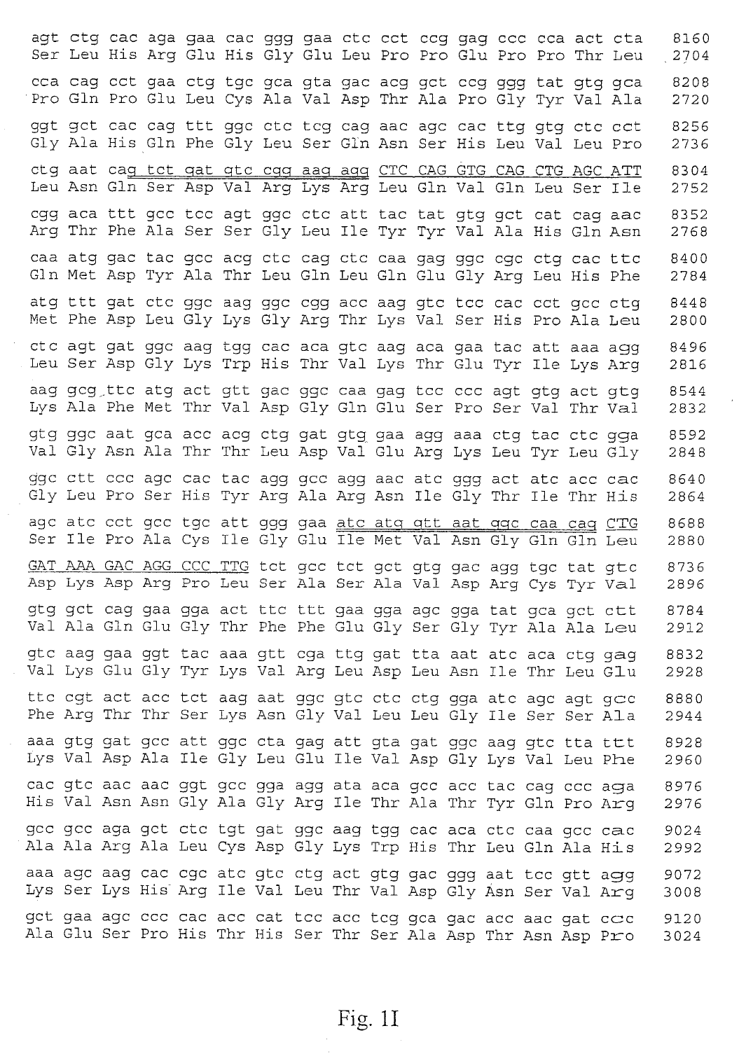

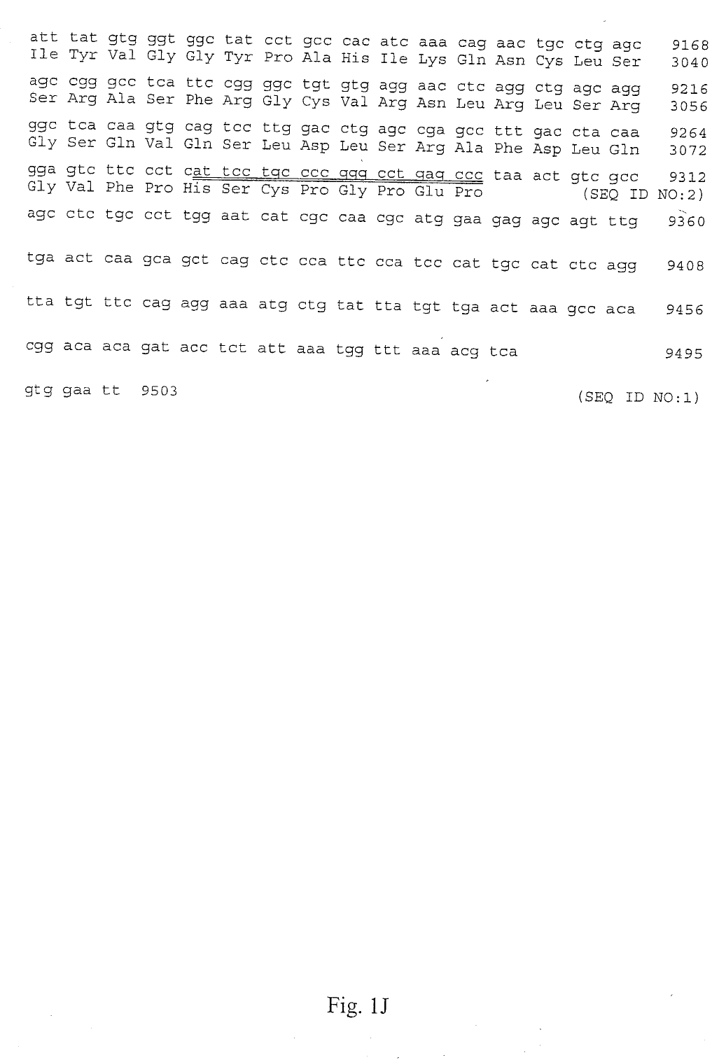

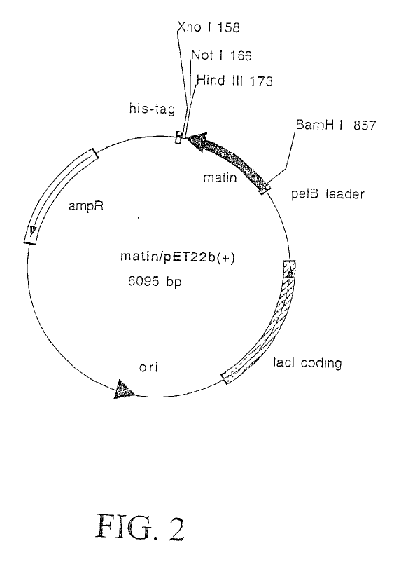

The nucleotide (SEQ ID NO: 1) and amino acid (SEQ ID NO: 2) sequences for the α1 chain of laminin (GenBank Ace. No. NM_{—}008480) are shown in FIG. 1. The sequence encoding Matin (globular domain 1, or the G1 domain, extending approximately from nucleotide 6442 to nucleotide 7062) was amplified by PCR from the plasmid FBssrAi using the forward primer 5′-CGG-GAT-CCT-AGA-GAC-TGC-ATC-CGC-GCC-TAT-3′ (SEQ ID NO: 3), and the reverse primer was 5′-CCC-AAG-CTT-TAC-TAT-CTG-CGT-CAC-GGT-GGG-3′ (SEQ ID NO: 4) (underlined portions of the primer represent laminin sequence). The resulting CDNA fragment was digested with BamHI and HindIII and ligated into predigested pET22b(+) (Novagen, Madison, Wis., USA). The construct is shown in FIG. 2. The ligation placed Matin in-frame with the pelB leader sequence, allowing for periplasmic localization and expression of soluble protein. The 3′ end of the sequence was ligated in-frame with the polyhistidine tag sequence.

Plasmid constructs encoding Matin were first transformed into E. coli HMS 174 (Novagen, Madison, Wis., USA) and then transformed into BL21 for expression (Novagen, Madison, Wis., USA). Overnight bacterial culture was used to inoculate a 500 ml culture in LB medium (Fisher Scientific, Pittsburgh, Pa., USA). This culture was grown for approximately 4 hours until the cells reached an OD₆₀₀ of 0.6. Protein expression was then induced by addition of IPTG to a final concentration of 1 mM. After a 2-hour induction, cells were harvested by centrifugation at 5,000×g and lysed by resuspension in 6 M guanidine, 0.1 M NaH₂PO₄, 0.01 M Tris-HCl, pH 8.0. Resuspended cells were sonicated briefly, and centrifuged at 12,000×g for 30 minutes. The supernatant fraction was passed over a 5 ml Ni-NTA agarose column (Qiagen, Hilden, Germany) 4-6 times at a speed of 2 ml per minute. Non-specifically bound protein was removed by washing with both IO mM and 25 mM imidazole in 8 M urea, 0.1 M NaH₂PO₄, 0.01 M Tris-HCl, pH 8.0. Matin protein was eluted from the column with increasing concentrations of imidazole (50 mM, 125 mM, and 250 mM) in 8 M urea, 0.1 M NaH₂PO₄, 0.01 M Tris-HCl, pH 8.0. The eluted protein was dialyzed twice against PBS at 4° C. A portion of the total protein precipitated during dialysis. Dialyzed protein was collected and centrifuged at approximately 3,500×g and separated into insoluble (pellet) and soluble (supernatant) fractions.

E. coli-expressed Matin was isolated predominantly as a soluble protein and SDS-PAGE analysis revealed a monomeric band at about 30 kDa. The eluted fractions containing this band were used in the following experiments. Protein concentration in each fraction was determined by the BCA assay (Pierce Chemical Co., Rockford, Ill., USA) and quantitative SDS-PAGE analysis using scanning densitometry.

### Example 2

### Matin Inhibits Endothelial Cell Proliferation

The anti-proliferative effect of Matin on C-PAE cells was examined by the methylene blue staining assay using E. coli produced soluble protein.

Cell lines and culture. PC-3 (human prostate adenocarcinoma cell line) and C-PAE (bovine pulmonary arterial endothelial cell line) cells were obtained from American Type Culture Collection. The C-PAE cell lines were maintained in DMEM (Life Technologies/Gibco BRL, Gaithersburg, Md., USA) supplemented with 10% fetal calf serum (FCS), 100 units/ml of penicillin, and 100 mg/ml of streptomycin.

Proliferation assay. C-PAE cells were grown to confluence in DMEM with 10% FCS and kept contact-inhibited for 48 hours. C-PAE cells were used between the second and fourth passages. PC-3 cells were used as non-endothelial controls in this experiment. Cells were harvested by trypsinization (Life Technologies/Gibco BRL, Gaithersberg, Md., USA) at 37° C. for 5 minutes. A suspension of 12,500 cells in DMEM with 0.1% FCS was added to each well of a 24-well plate coated with 10 μg/ml fibronectin. The cells were incubated for 24 hours at 37° C. with 5% CO₂ and 95% humidity. Medium was removed and replaced with DMEM containing 20% FCS. Unstimulated control cells were incubated with 0.1% FCS.

Using the methylene-blue staining method, 7000 cells were plated into each well of a 96-well plate, and treated as described above. Cells were then counted using the method of Oliver et al. (Oliver, M. H. et al., 1989, J. Cell. Sci. 92:513-518). After 48 hours of treatment, all wells were washed with 100 μl of PBS, and the cells fixed with 10% formalin in neutral-buffered saline (Sigma Chemical Co., St. Louis, Mo., USA). The cells were then stained with 1% methylene blue (Sigma) in 0.01M borate buffer, pH 8.5. Wells were washed with 0.01M borate buffer, and the methylene blue extracted from the cells with 0.1N HCl/ethanol, and the absorbance measured in a microplate reader (Bio-Rad, Hercules, Calif., USA) at 655 nm. Polymyxin B (Sigma) at a final concentration of 5 μg/ml was used to inactivate endotoxin (Liu, S. et al., 1997, Clin. Biochem. 30:455-463).

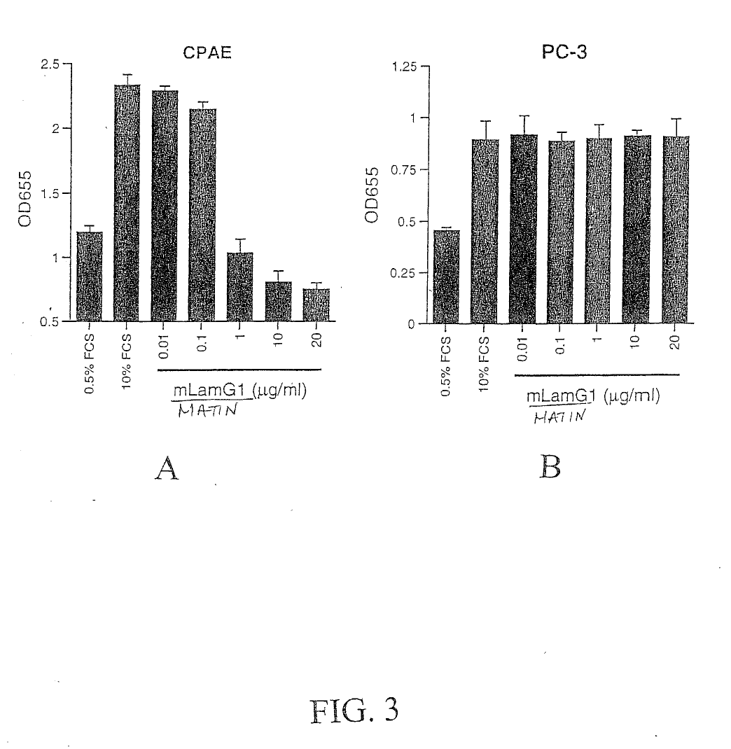

The results are shown in FIGS. 3A and 3B, which are a pair of histograms showing the effect of increasing amounts of Matin on the uptake of dye by C-PAE cells relative to PC-3 cells. Absorbance at OD₆₅₅ is shown on the y-axis. “0.5% FCS” represents the 0.5% FCS-treated (unstimulated) control, and “10% FCS” is the 10% FCS-treated (stimulated) control. The remaining bars represent treatments with increasing concentrations of Matin. Matin inhibited FCS-stimulated proliferation of C-PAE cells in a dose-dependent manner. The difference between the mean value of the cell number in the Matin treatment versus the control was significant in the 0.1-10.0 μg/ml range, with p<0.05. When PC-3 cells were treated with Matin, no inhibitory effect was observed. In C-PAE cells, dye uptake dropped off to the level seen in unstimulated cells at a Matin treatment level of about 0.1 μg/ml. Each bars represents the mean of the relative absorbance units at 655 nm±the standard error of the mean for triplicate wells. This endothelial cell specificity indicates that Matin is likely an effective anti-angiogenic agent.

### Example 3

### Matin Induces Endothelial Cell Apoptosis

Annexin V-FITC assay. In the early stage of apoptosis, translocation of the membrane phospholipid PS from the inner surface of plasma membrane to outside is observed (van Engeland, M. et al., 1998, Cytometry 31:1-9; Zhang, G. et al., 1997, Biotechniques 23:525-531; Koopman, G. et al. 1994, Blood 84:1415-1420). Externalized PS can be detected by staining with a FITC conjugate of Annexin V that has a naturally high binding affinity to PS (van Engeland, supra). Apoptosis of endothelial cells upon treatment with Matin was therefore evaluated using annexin V-FITC labeling.

C-PAE cells (0.5×10⁶ per well) were seeded onto a 6-well plate in 10% FCS supplemented DMEM. The next day, fresh medium containing 10% FCS was added with either 80 ng/ml of TNF-α (positive control) or Matin ranging from 0.02 to 20 μg/ml. Control cells received an equal volume of PBS. After 18 hours of treatment, medium containing floating cells was collected, and attached cells were trypsinized and centrifuged together with floating cells at 3,000×g. The cells were then washed in PBS and resuspended in binding buffer (10 mM HEPES/NaOH, pH 7.4, 140 mM NaCl, 2.5 mM CaCl₂). Annexin V-FITC (Clontech, Palo Alto, Calif., USA) was added to a final concentration of 150 ng/ml, and the cells were incubated in the dark for 10 minutes. The cells were washed again in PBS and resuspended in binding buffer. Annexin V-FITC labeled cells were counted using a FACStar Plus flow cytometer (Becton-Dickinson, Waltham, Mass., USA). For each treatment, 15,000 cells were counted and stored in listmode. This data was then analyzed using Cell Quest software (Becton-Dickinson, Waltham, Mass., USA).

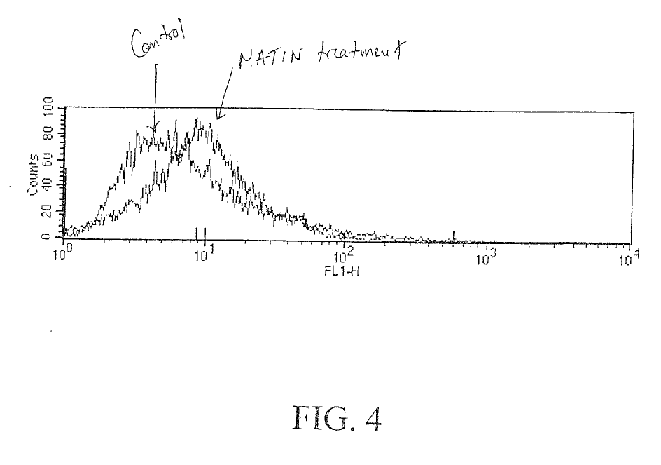

The results are shown in FIG. 4, which is a plot showing annexin fluorescence activity. After 18 hours of treatment with Matin at 20 μg/ml, a distinct shift of peak annexin fluorescence was observed. The shift in fluorescence intensity was similar for Matin at 20 μg/ml and the positive control TNF-α (80 ng/ml). Matin at 2 μg/ml also showed a mild shift in annexin fluorescence intensity, but concentrations below 0.2 μg/ml did not demonstrate any annexin V positivity. This shift of peak intensity was not observed when nonendothelial cells (PC-3) were used.

Caspase-3 assay. Caspase-3 (CPP32) is an intracellular protease activated at the early stage of apoptosis, and initiates cellular breakdown by degrading structural and DNA repair proteins (Casciola-Rosen, L. et al., 1996, J. Exp. Med. 183:1957-1964; Salvesen, G. S. et al., 1997, Cell 91:443-446). The protease activity of Caspase-3 was measured spectrophotometrically by detection of the chromophore (p-nitroanilide) cleaved from the labeled substrate (DEVD-pNA).

C-PAE cells or PC-3 cells (0.5×10⁶ per well) were plated onto a 6-well plate precoated with fibronectin (10 μg/ml) in DMEM supplemented with 10% FCS, and incubated overnight. The next day, the medium was replaced with DMEM containing 2% FCS and then incubated overnight at 37° C. Then cells were then stimulated with bFGF (3 ng/ml) in DMEM supplemented with 2% FCS, and also containing either TNF-α (80 ng/ml, positive control) or Matin (10 μg/ml), and incubated for 24 hours. Controls received PBS buffer. After 24 hours, the supernatant cells were collected, and attached cells were trypsinized and combined with the supernatant cells. Cells were counted and resuspended in cell lysis buffer (Clontech, Palo Alto, Calif., USA) at a concentration of 4×10⁷ cells/ml. The rest of the protocol followed the manufacturer's instructions (Clontech, Palo Alto, Calif., USA). A specific inhibitor of Caspase-3, DEVD-fmk (Asp-Glu-Val-Asp-fluoromethyl ketone) was used to confirm the specificity of the assay. The absorbance was measured in a microplate reader (Bio-Rad, Hercules, Calif., USA) at 405 nm.

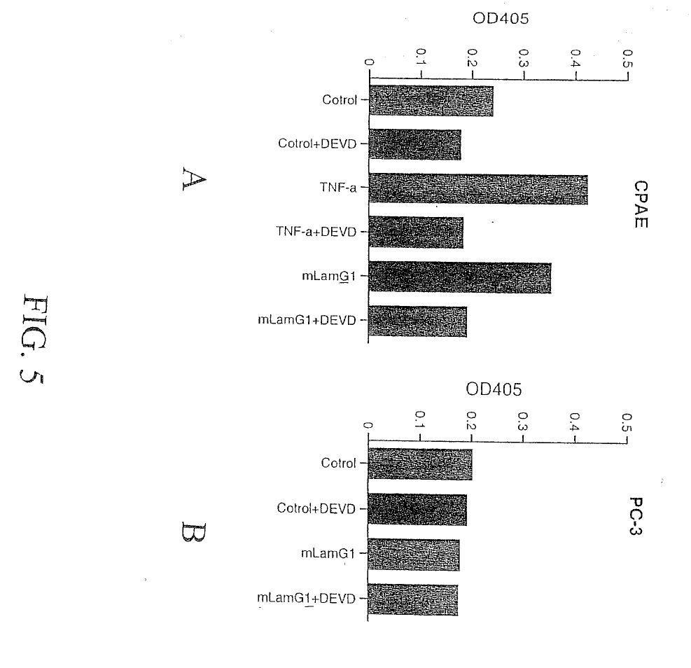

The results are shown in FIGS. 5A and 5B, which are a pair of histograms showing the amount of Caspase-3 activity as a function of absorbance at OD₄₀₅ (y-axis) for C-PAE cells (FIG. 5A) and PC-3 cells (FIG. 5B) under various treatments (x-axis).

C-PAE cells treated with 20 μg/ml Matin exhibited a 1.6-fold increase in Caspase-3 activity, whereas the positive control TNF-α gave a comparable (1.7-fold) increase compared with control. A specific inhibitor of Caspase-3, DEVD-fmk, decreased the protease activity to baseline indicating that the increase in the measured activity was specific for Caspase-3. In nonendothelial PC-3 cells, there was no difference in Caspase-3 activity between control and Matin-treated cells.

MTT Assay. The pro-apoptotic activity of Matin was examined in C-PAE cells. Cell viability was assessed by MTT (3-(4, 5-dimethylthiazol-2-yl)-2, 5-diphenyl tetrasolium bromide) assay (Sugiyama, H. et al., 1998, Kidney Int. 54:1188-1196). This assay is a quantitative calorimetric analysis for cell survival based on the ability of living cells to cleave the tetrasolium ring in active mitochondria. C-PAE cells (7,000 cells per well) were plated to a 96-well plate in 10% FCS containing DMEM. The next day, either TNF-α (positive control, 80 ng/ml), or varying concentrations of Matin were added to the wells and incubated for 24 hours. MTT solution (5 mg/ml; CHEMICON International, Temecula, Calif., USA) was then added to the wells at a rate of 10 μl/well and incubated at 37° C. for 4 hours. Acid-isopropanol was added and mixed thoroughly. The absorbance was measured in a microplate reader (Bio-Rad, Hercules, Calif., USA) at 590 nm.

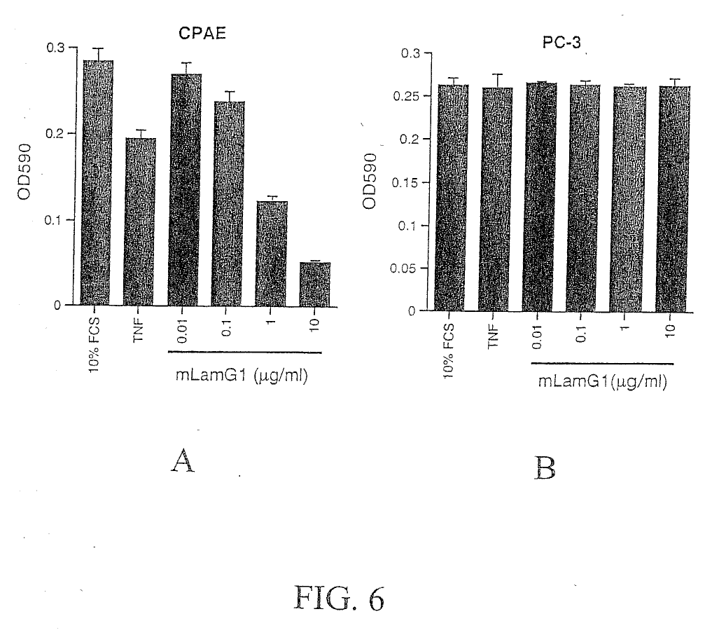

The results are shown in FIGS. 6A and 6B, which are a pair of histograms showing cell viability (as a function of OD₅₉₀, y-axis) at increasing concentrations of Matin (x-axis). Each point represents the mean +/− the standard error of the mean for triplicate wells.

Matin decreased cell viability in a dose-dependent manner. At 10 μg/ml, Matin decreased the cell viability by about 80% compared to controls. No inhibitory effect was observed in Matin-treated PC-3 cells.

### Example 4

### Matin Inhibits Tumor Growth in vivo

Five million PC-3 cells were harvested and injected subcutaneously on the back of 7- to 9-week-old male athymic nude mice. The tumors were measured using Vernier calipers and the volume was calculated using the standard formula width²×length×0.52. The tumors were allowed to grow to about 100 mm³, and animals were then divided into groups of 5 or 6 mice. Matin or nephrin was intraperitoneally injected daily (20 mg/kg) for 10 days in sterile PBS to their respective experimental group. The control group received vehicle injection (either BSA or PBS). Tumor volume was calculated every 2 or 3 days over 10 days. Nephrin is a non-collagen-derived protein which was used as a control. The nephrin was expressed in pET22b, as for Matin.

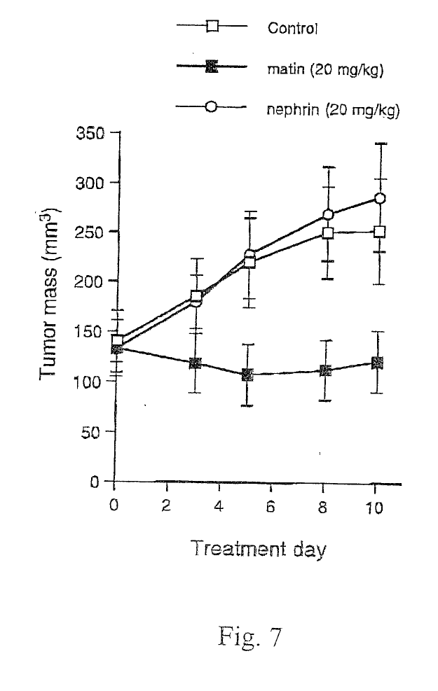

The results are shown in FIG. 7, which is a graph showing tumor size in mm³ (y-axis) against days of treatment (x-axis) for the PBS control (□), 20 mg/kg Matin (▪) and 20 mg/kg nephrin (◯). Matin, produced in E. coli, significantly inhibited the growth of PC-3 human prostate tumors (FIG. 7).

All references, patents, and patent applications are incorporated herein by reference in their entirety. While this invention has been particularly shown and described with references

to preferred embodiments thereof, it will be understood by those of ordinary skill in the art that various changes in form and details may be made therein without departing from the spirit and scope of the invention as defined by the appended claims.

What is claimed is:

1. An isolated Matin, wherein Matin is an isolated protein of the G1 domain of the a chain of laminin, or a fragment, analog, derivative or mutant thereof, wherein the protein, fragment, analog, derivative or mutant thereof has anti-angiogenic activity.

2. The Matin of claim 1, wherein the Matin is the G1 domain of the α1 chain of mouse laminin.

3. An isolated protein of SEQ ID NO: 2, or a fragment, analog, derivative or mutant thereof, wherein the protein, fragment, analog, derivative or mutant has anti-angiogenic activity.

4. An isolated protein or peptide having 90% or greater sequence identity with SEQ ID NO: 2, wherein the protein or peptide has anti-angiogenic activity.

5. An isolated protein or peptide having 80% or greater sequence identity with SEQ ID NO: 2, wherein the protein or peptide has anti-angiogenic activity.

6. The protein, fragment, analog, derivative or mutant of claim 1, wherein the protein, fragment, analog, derivative or mutant is a monomer.

7. A multimer of the protein, fragment, analog, derivative or mutant of claim 1, wherein the multimer has anti-angiogenic activity.

8. A chimeric protein comprising one or more protein, fragment, analog, derivative or mutant of claim 1, wherein the chimeric protein has anti-angiogenic activity.

9. The chimeric protein of claim 8, further comprising at least one protein molecule selected from the group consisting of: Vascostatin or fragments thereof, arresten or fragments thereof, canstatin or fragments thereof, tumstatin or fragments thereof, endostatin or fragments thereof, angiostatin or fragments thereof, restin or fragments thereof, apomigren or fragments thereof, or other anti-angiogenic proteins or fragments thereof.

10. The use of the protein, fragment, analog, derivative, mutant, multimer or chimeric protein of claim 1 in the preparation of a medicament for treating a disorder involving inhibiting angiogenesis in a tissue.

11. The use according to claim 10, wherein the disorder is tumor growth.

12. The use of the protein, fragment, analog, derivative, mutant, multimer or chimeric protein of claim 1 in the preparation of a medicament for treating a disorder by promoting or inducing endothelial cell apoptosis in a tissue.

13. The use of claim 10, wherein the angiogenesis is inhibited by inhibiting one or more of the following: endothelial cell proliferation, endothelial cell migration, or endothelial cell tube formation.

14. A pharmaceutical composition comprising one or more of the proteins, fragments, analogs, derivatives, mutants, multimers or chimeric proteins of claim 1.

15. The pharmaceutical composition of claim 14, and a pharmaceutically-compatible carrier.

16. The pharmaceutical composition of claim 15, further comprising at least one protein molecule selected from the group consisting of: Vascostatin or fragments thereof, arresten or fragments thereof, canstatin or fragments thereof, tumstatin or fragments thereof, endostatin or fragments thereof, angiostatin or fragments thereof, restin or fragments thereof, apomigren or fragments thereof, or other anti-angiogenic proteins, or fragments thereof.

17. A method of treating a subject comprising using the pharmaceutical composition of claim 14.

18. A method for inhibiting angiogenic activity in mammalian tissue, the method comprising contacting the tissue with the composition of claim 14.

19. The method of claim 18, wherein the angiogenic activity is characteristic of a disease selected from the group comprising angiogenesis-dependent cancers, benign tumors, rheumatoid arthritis, diabetic retinopathy, fibrosis, psoriasis, ocular angiogenesis diseases, Osler-Webber Syndrome, myocardial angiogenesis, plaque neovascularization, telangiectasia, hemopheliac joints, angiofibroma, wound granulation, intestinal adhesions, atherosclerosis, seleroderma, hypertrophic scars, cat scratch disease, Heliobacter pylori ulcers, dialysis graft vascular access stenosis, contraception and obesity.

20. The method of claim 19, wherein the disease is cancer.

21. A method of using the composition of claim 14 to inhibit a disease characterized by angiogenic activity, the method comprising administering to a patient with the disease, the composition in conjunction with radiation therapy, chemotherapy, or immunotherapy.

22. An isolated polynucleotide encoding the protein, fragment, analog, derivative or mutant of claim 1, wherein the protein, fragment, analog, derivative or mutant has anti-angiogenic activity.

23. The isolated polynucleotide of claim 17, wherein the polynucleotide is SEQ ID NO: 1.

24. An isolated polynucleotide having 90% or greater sequence identity with SEQ ID NO: 1, wherein the isolated polynucleotide encodes a polypeptide having anti-angiogenic activity.

25. An isolated polynucleotide having 85% or greater sequence identity with SEQ ID NO: 1, wherein the isolated polynucleotide encodes a polypeptide having anti-angiogenic activity.

26. The isolated polynucleotide of claim 17, wherein the polynucleotide is operably linked to an expression control sequence.

27. A host cell transformed with the polynucleotide of claim 21.

28. The host cell of claim 22, where the cell is selected from the group comprising bacterial, yeast, mammalian, insect or plant cells.

29. An antibody that specifically binds to the isolated protein, fragment, analog, derivative, mutant, multimer or chimeric protein of claim 1.

30. A process for producing a protein encoded by the polynucleotide of claim 22, wherein the process comprises:
(a) growing a culture of a host cell transformed with the polynucleotide of claim 22, where the host cell is selected from the group comprising bacterial, yeast, mammalian, insect or plant cells; and
(b) purifying the protein from the culture;
thereby producing the protein encoded by the polynucleotide of claim 22.

31. An isolated polynucleotide produced according to the process of:
(a) preparing one or more polynucleotide probes that hybridize under conditions under moderate stringency to the polynucleotide of claim 22;
(b) hybridizing said probe(s) to mammalian DNA; and
(c) isolating the DNA polynucleotide detected with the probe(s);
wherein the nucleotide sequence of the isolated polynucleotide corresponds to the nucleotide sequence of the polynucleotide of claim 22.

32. A method for providing a mammal with an anti-angiogenic protein, the method comprising introducing mammalian cells into a mammal, said mammalian cells having been treated in vitro to insert therein the polynucleotide of claim 22, and expressing in vivo in said mammal a therapeutically effective amount of the anti-angiogenic protein in an amount sufficient to inhibit angiogenic activity in the mammal.

33. The method of claim 32 wherein the expression of the anti-angiogenic protein is transient expression.

34. The process of claim 32, wherein the cells are chosen from the group consisting of: blood cells, TIL cells, bone marrow cells, vascular cells, tumor cells, liver cells, muscle cells, fibroblast cells.

35. The process of claim 34, wherein the polynucleotide is inserted into the cells by a viral vector.


SEQUENCE LISTING (12 sequences)

SEQ ID NO: 1 - DNA, 9503 bp, Mus musculus
  CDS (49)...(9300)
agcacgggcgagaccttccccaggagcgcagggagcggcggcgacaacatgcgcggcagcggcacgggagccgcgctcctggtgctcctggcctcggtgctctgggtcaccgtgcggagccagcagagaggcttgttccctgccattctcaacctggccaccaatgcccacatcagcgccaatgctacctgtggagagaaggggcctgagatgttctgcaaactcgtggagcacgtgccgggccggcctgttcgacacgcccaatgccgggtctgtgacggtaacagtacgaatcctagagagcgccatccgatatcacacgcaatcgatggcaccaacaactggtggcagagccccagtattcagaatgggagagagtatcactgggtcactgtcaccctggacttacggcaggtctttcaagttgcatacatcatcattaaagctgccaatgcccctcggcctggaaactggattttggagcgctccgtggatggcgtcaagttcaaaccctggcagtactatgccgtcagcgatacagagtgtttgacccgctacaaaataactccacggcggggacctcccacttacagagcagacaacgaagtcatctgcacctcgtattattcaaagctggtgccacttgaacatggagagattcacacatcactcatcaatggcagacccagcgctgacgacccctcaccccagttgctggaattcacctcagcacggtacattcgccttcgtcttcagcgcatcagaacactcaacgcagacctcatgacccttagccatcgggacctcagagaccttgaccccattgtcacaagacgttattactattcgataaaagacatttccgttggaggcatgtgcatttgctacggccatgccagcagctgcccgtgggatgaagaagcaaagcaactacagtgtcagtgtgaacacaatacgtgtggcgagagctgcgacaggtgctgtcctggctaccatcagcagccctggaggcccggaaccatttcctccggcaacgagtgtgaggaatgcaactgtcacaacaaagccaaagattgttactatgacagcagtgttgcaaaggagaggagaagcctgaacactgccgggcagtacagtggaggaggggtttgtgtcaactgctcgcagaataccacagggatcaactgtgaaacctgtatcgaccagtattacagacctcacaaggtatctccttatgatgaccacccttgccgtccctgtaactgtgaccctgtggggtctctgagttctgtctgtatcaaggatgaccgccatgccgatttagccaatggaaagtggccaggtcagtgtccatgtaggaaaggttatgctggagataaatgtgaccgctgccagtttggctaccggggtttcccaaattgcatcccctgtgactgcaggactgtcggcagcctgaatgaggatccatgcatagagccgtgtctttgtaagaaaaatgttgagggtaagaactgtgatcgctgcaagccaggattctacaacttgaaggaacgaaaccccgagggctgctccgagtgcttctgcttcggtgtctctggtgtctgtgacagcctcacgtggtccattagtcaggtgaccaatatgtcagggtggctggtcactgacttgatgagcacaaataagatccggtcccagcaagatgtcctgggtgggcaccgtcagatcagcatcaacaacacggcggtcatgcagaggctgacttccacttactactgggcagctcctgaggcctacctcggaaacaagctgacagcatttggcggtttcctgaaatacactgtgtcttacgacattccagtggagacggtggacagtgacctcatgtctcatgctgacatcatcatcaaggggaatgggctcaccataagcacaagagctgagggcctgtccttgcaaccctatgaggaatacttcaacgtggttagacttgtgcctgagaacttccgggactttaataccagaagggagattgaccgtgaccagctgatgactgtcctggccaatgtgacacatctcttgatcagagccaattataattctgctaaaatggcgctctataggctggattctgtctctctggacatagcaagccctaatgctatagacttggcagtggctgctgatgtggaacactgtgaatgtccccaaggctacacggggacctcctgtgaggcctgcctccctggctattatcgagtggacgggatactctttggaggaatctgtcagccctgcgagtgccacgggcatgcatccgagtgtgacattcatggaatttgctctgtgtgtacacacaacaccacgggggatcactgtgagcagtgcctgcctggcttctatgggacaccttcacgtgggaccccaggagactgccagccttgtgcctgccctctctccattgactctaacaatttcagccctacctgccacctcactgatggagaggaagtggtttgtgaccaatgtgccccgggttactcaggatcctggtgtgagagatgtgcagatggttactatggaaacccaaccgtgccagggggaacctgtgtaccatgcaactgcagtggcaatgttgatcccttggaggctggccactgtgactctgtcacgggggaatgcctgaagtgcttatggaacacagacggtgcccattgtgagaggtgtgcagatggcttctatggagatgccgtgactgccaaaaactgccgagcctgtgactgccacgagaatggctccctttctggcgtctgccatctggagactggactgtgtgactgcaaacctcacgtgacaggacagcagtgtgaccagtgcctgtctggctactacgggttggacacggggcttggctgtgtgccctgtaactgcagtgtggaaggctctgtatctgacaactgcacggaggaaggacagtgtcactgtggaccaggtgtctctgggaaacagtgtgacaggtgttcacatggtttctatgcattccaggatggcggctgcacaccctgtgactgtgctcatacccagaataactgtgaccccgcctctggagagtgtctctgcccgcctcacacgcaggggctgaagtgtgaggagtgtgaagaggcatactggggtctggacccggagcaggggtgccaggcttgcaattgcagtgctgtgggctccacgagtgcccagtgtgatgttctctctggccactgcccctgcaaaaaagggtttggtgggcagagctgccatcagtgttccttaggctacagaagttttcctgactgtgtcccctgtggctgtgacctgagggggacactgcctgacacctgtgacctggaacagggtctctgcagctgctcagaggacagtggtacctgctcctgcaaggagaatgtcgtgggcccccagtgcagtaagtgccaagccggcacctttgccttgcgaggggacaaccctcaaggctgcagcccctgcttctgcttcggtctgtcgcagctctgctcagagttggagggttacgtgaggactctgataactctagcctccgatcagcccctcctgcatgtggtttcacagagcaacctcaagggcacaatcgaaggcgtgcatttccagcctcctgacaccttgctggacgcagaggctgtccgccagcatatctatgcagagccattttactggcggctaccaaagcagttccagggagaccagctcttggcctatggtgggaaactccagtacagtgtggctttctactctacacttggcaccggaacatccaattatgagcctcaagtcctcatcaaaggaggtcgggccaggaagcacgtcatttatatggatgccccagcgcctgagaatggagtgagacaggattacgaagtgcagatgaaagaggaattctggaaatattttaactccgtgtctgagaaacacgtcacacactctgattttatgtctgttctcagcaatattgactacatcctcatcaaagcatcatacggccagggactgcagcagagcagaattgccaacatttccatggaggttggccggaaagctgtcgagctgcccgctgagggcgaggcggcattgctgttggagctctgtgtctgtcctcctggcaccgcaggacactcctgtcaggactgtgctcctgggtactacagagaaaagctcccagaaagtggtggcaggggaccccgccctctgctggctccttgtgtgccctgcaattgcaacaaccacagtgatgtctgtgaccccgaaactggaaagtgcctgagctgcagggaccacacatccggggaccactgtgagctgtgtgcttctggctactatgggaaggtgactggactgcctggagactgtaccccgtgcacctgtcctcatcaccctcctttcagtttcagccccacttgtgtcgtggaaggtgacagtgatttccggtgcaatgcctgcctccccggctatgaaggacagtactgtgaaaggtgctctgcaggctatcacggcaaccctcgagcagcaggtggtagctgccaaacgtgtgattgcaacccccaaggctctgtccacagtgactgtgaccgtgcatccgggcagtgtgtctgcaagccaggagctacagggctccactgtgagaaatgcctgccgagacacatcctgatggagagcgactgtgtttcctgtgatgatgactgtgtgggtcctttgctgaacgacctggattctgttggtgatgccgttctgtctctgaacctcacgggcgtttcccctgctccctatggaatcctggaaaatctggaaaatacaactaaatatttccagaggtatttaataaaggaaaatgccaagaagattcgagcagagatccagctcgaagggattgcagagcaaacagaaaatctgcaaaaggagctcaccagagtgttagcacgccatcagaaggtgaacgctgaaatggaaagaacttccaatgggactcaagccctggccacgttcattgagcagctacatgcaaacatcaaagaaatcacagaaaaggtggcaacgttgaaccagacggcgcgtaaagatttccagccacccgtgtctgcccttcagagcatgcaccagaacatttcgtctctgctgggactcatcaaggaaaggaatttcacagagatgcagcagaatgctacccttgagctcaaggctgctaaagacttattgtcacggattcagaaaaggtttcagaagcctcaggaaaagttgaaggcattgaaggaggccaacagcctcctttccaaccacagtgaaaaactgcaggctgctgaggagctccttaaggaagctggaagcaagacccaggagagcaacctcctgctgctccttgtcaaggccaacctgaaagaggaattccaggagaaaaagctgcgtgttcaagaagaacaaaatgtgacctcagagctcattgccaagggtagagaatgggtggatgctgccgggactcacacagctgctgcacaagacaccctaacacagctggagcatcaccgagatgaactccttctgtgggccagaaaaatcaggagccacgtagatgacctcgtcatgcagatgtccaaacgaagagcccgtgacctggtccacagggcagagcagcatgcctctgagctgcagagcagggcaggagctttggacagagaccttgaaaatgttagaaacgtgtctttgaatgccaccagtgcggcacacgtccacagcaacatccagacactgacagaggaagctgagatgctggctgctgatgctcacaagacggcgaataagacagacttgatctccgaatccctggcttctcgggggaaagcagtccttcagcgctcgtcccggtttctaaaggaaagtgtcggtaccaggaggaagcagcaaggcattacgatgaagctggatgagttgaaaaacttaacgagtcaatttcaggagagcgtggataacattacgaagcaggccaacgactcccttgcgatgcttagagaaagccctggaggtatgagagagaagggcaggaaagccagagagctggcggcagcagccaacgagagtgcggtgaagacactggaggatgtgctggctttgagcctgagggtcttcaatacatcagaggacctgtccagagtgaatgccacagtccaggagacaaacgaccttctgcataactccacgatgaccactctgttggctggaagaaaaatgaaagacatggaaatgcaagccaaccttttattggatcgattgaaacctttgaaaaccctggaggagaacctgagcagaaacctgtcggagatcaagctgctgatcagccgggcccggaaacaagcggcgtcgatcaaagtcgccgtgtctgcagacagagactgcatccgcgcctatcagcctcagacttcatctaccaattacaacaccttgatcctgaacgtgaagacgcaggagcccgacaacctcctcttctacctcggcagcagcagcagttctgactttctcgcagtggagatgcggcgggggaaggtcgcctttctctgggacctgggctccgggtccacaaggttggaattcccagaggtctccatcaataacaacagatggcacagcatctacataaccaggtttggaaacatggggtccctgagtgtaaaggaagcaagcgctgccgagaacccaccggtcaggacaagcaaatctcctggaccgtcgaaggttctggacataaacaattcaacgctgatgtttgttggagggctcggaggtcagatcaagaaatccccggctgtgaaggttactcattttaagggctgcatgggagaggccttcttgaatggcaaatcgattggcctgtggaattacatcgagagagaggggaagtgcaatggctgctttggaagctcccagaacgaagattcctccttccatttcgatggaagcgggtacgccatggtggagaagacgctccggcccaccgtgacgcagatagtaattctcttcagcaccttctccccgaatggccttcttttctacctggcttcaaacggcaccaaggacttcctatccatcgagctggtccgtggcagggtcaaagtgatggtggacctagggtcaggacccctcactcttatgacagacaggcggtataacaacggaacctggtataaaatcgccttccagcggaaccggaagcaaggactgctagctgtcttcgatgcatatgacaccagtgacaaggagacaaagcaaggagagactccaggagccgcttccgacctcaatcggctggagaaagacctgatttacgtgggtggattgcctcattctaaggctgtgaggaaaggggtcagcagcagaagctatgtgggctgtatcaagaacctagagatatccaggtccacctttgatttgctgagaaattcctacggagtgagaaaaggctgcgcactggagcctatccagagtgtgagtttcctgagaggcggctatgtggagatgccacccaagtctctctcacctgagtcatccctgctggccacattcgccaccaagaacagcagcggaatcctcctggttgccctgggcaaggatgcggaggaggctggtggggctcaggcacatgtgcccttcttttccatcatgctgcttgagggacgaattgaagtgcatgtcaactctggggacgggaccagtctgaggaaggccctcctgcatgcccccaccggctcctacagtgatggacaggaacactccatctccctggttaggaatcggagagttatcaccatacaagtggatgagaacagtcccgtagaaatgaagttgggtccattaacagaaggaaagacgatcgacatatccaacctgtacatagggggacttccggaggacaaggcgaccccgatgctcaagatgcggacttcgttccatgggtgtattaaaaacgtggtccttgacgctcaacttttggacttcacccatgcgactggctctgagcaagtagagctggacacatgcttgctggcagaagagcccatgcagagtctgcacagagaacacggggaactccctccggagcccccaactctaccacagcctgaactgtgcgcagtagacacggctccggggtatgtggcaggtgctcaccagtttggcctctcgcagaacagccacttggtgctccctctgaatcagtctgatgtccggaagaggctccaggtgcagctgagcattcggacatttgcctccagtggcctcatttactatgtggctcatcagaaccaaatggactacgccacgctccagctccaagagggccgcctgcacttcatgtttgatctcggcaagggccggaccaaggtctcccaccctgccctgctcagtgatggcaagtggcacacagtcaagacagaatacattaaaaggaaggcgttcatgactgttgacggccaagagtcccccagtgtgactgtggtgggcaatgcaaccacgctggatgtggaaaggaaactgtacctcggaggccttcccagccactacagggccaggaacatcgggactatcacccacagcatccctgcctgcattggggaaatcatggttaatggccaacagctggataaagacaggcccttgtctgcctctgctgtggacaggtgctatgtcgtggctcaggaaggaactttctttgaaggaagcggatatgcagctcttgtcaaggaaggttacaaagttcgattggatttaaatatcacactggagttccgtactacctctaagaatggcgtcctcctgggaatcagcagtgccaaagtggatgccattggcctagagattgtagatggcaaggtcttatttcacgtcaacaacggtgccggaaggataacagccacctaccagcccagagccgccagagctctctgtgatggcaagtggcacacactccaagcccacaaaagcaagcaccgcatcgtcctgactgtggacgggaattccgttagggctgaaagcccccacacccattccacctcggcagacaccaacgatcccatttatgtgggtggctatcctgcccacatcaaacagaactgcctgagcagccgggcctcattccggggctgtgtgaggaacctcaggctgagcaggggctcacaagtgcagtccttggacctgagccgagcctttgacctacaaggagtcttccctcattcctgccccgggcctgagccctaaactgtcgccagcctctgcccttggaatcatcgccaacgcatggaagagagcagtttgtgaactcaagcagctcagctcccattcccatcccattgccatctcaggttatgtttccagaggaaaatgctgtatttatgttgaactaaagccacacggacaacagatacctctattaaatggtttaaaacgtcagtggaatt

SEQ ID NO: 2 - protein, 3084 aa, Mus musculus
MRGSGTGAALLVLLASVLWVTVRSQQRGLFPAILNLATNAHISANATCGEKGPEMFCKLVEHVPGRPVRHAQCRVCDGNSTNPRERHPISHAIDGTNNWWQSPSIQNGREYHWVTVTLDLRQVFQVAYIIIKAANAPRPGNWILERSVDGVKFKPWQYYAVSDTECLTRYKITPRRGPPTYRADNEVICTSYYSKLVPLEHGEIHTSLINGRPSADDPSPQLLEFTSARYIRLRLQRIRTLNADLMTLSHRDLRDLDPIVTRRYYYSIKDISVGGMCICYGHASSCPWDEEAKQLQCQCEHNTCGESCDRCCPGYHQQPWRPGTISSGNECEECNCHNKAKDCYYDSSVAKERRSLNTAGQYSGGGVCVNCSQNTTGINCETCIDQYYRPHKVSPYDDHPCRPCNCDPVGSLSSVCIKDDRHADLANGKWPGQCPCRKGYAGDKCDRCQFGYRGFPNCIPCDCRTVGSLNEDPCIEPCLCKKNVEGKNCDRCKPGFYNLKERNPEGCSECFCFGVSGVCDSLTWSISQVTNMSGWLVTDLMSTNKIRSQQDVLGGHRQISINNTAVMQRLTSTYYWAAPEAYLGNKLTAFGGFLKYTVSYDIPVETVDSDLMSHADIIIKGNGLTISTRAEGLSLQPYEEYFNVVRLVPENFRDFNTRREIDRDQLMTVLANVTHLLIRANYNSAKMALYRLDSVSLDIASPNAIDLAVAADVEHCECPQGYTGTSCEACLPGYYRVDGILFGGICQPCECHGHASECDIHGICSVCTHNTTGDHCEQCLPGFYGTPSRGTPGDCQPCACPLSIDSNNFSPTCHLTDGEEVVCDQCAPGYSGSWCERCADGYYGNPTVPGGTCVPCNCSGNVDPLEAGHCDSVTGECLKCLWNTDGAHCERCADGFYGDAVTAKNCRACDCHENGSLSGVCHLETGLCDCKPHVTGQQCDQCLSGYYGLDTGLGCVPCNCSVEGSVSDNCTEEGQCHCGPGVSGKQCDRCSHGFYAFQDGGCTPCDCAHTQNNCDPASGECLCPPHTQGLKCEECEEAYWGLDPEQGCQACNCSAVGSTSAQCDVLSGHCPCKKGFGGQSCHQCSLGYRSFPDCVPCGCDLRGTLPDTCDLEQGLCSCSEDSGTCSCKENVVGPQCSKCQAGTFALRGDNPQGCSPCFCFGLSQLCSELEGYVRTLITLASDQPLLHVVSQSNLKGTIEGVHFQPPDTLLDAEAVRQHIYAEPFYWRLPKQFQGDQLLAYGGKLQYSVAFYSTLGTGTSNYEPQVLIKGGRARKHVIYMDAPAPENGVRQDYEVQMKEEFWKYFNSVSEKHVTHSDFMSVLSNIDYILIKASYGQGLQQSRIANISMEVGRKAVELPAEGEAALLLELCVCPPGTAGHSCQDCAPGYYREKLPESGGRGPRPLLAPCVPCNCNNHSDVCDPETGKCLSCRDHTSGDHCELCASGYYGKVTGLPGDCTPCTCPHHPPFSFSPTCVVEGDSDFRCNACLPGYEGQYCERCSAGYHGNPRAAGGSCQTCDCNPQGSVHSDCDRASGQCVCKPGATGLHCEKCLPRHILMESDCVSCDDDCVGPLLNDLDSVGDAVLSLNLTGVSPAPYGILENLENTTKYFQRYLIKENAKKIRAEIQLEGIAEQTENLQKELTRVLARHQKVNAEMERTSNGTQALATFIEQLHANIKEITEKVATLNQTARKDFQPPVSALQSMHQNISSLLGLIKERNFTEMQQNATLELKAAKDLLSRIQKRFQKPQEKLKALKEANSLLSNHSEKLQAAEELLKEAGSKTQESNLLLLLVKANLKEEFQEKKLRVQEEQNVTSELIAKGREWVDAAGTHTAAAQDTLTQLEHHRDELLLWARKIRSHVDDLVMQMSKRRARDLVHRAEQHASELQSRAGALDRDLENVRNVSLNATSAAHVHSNIQTLTEEAEMLAADAHKTANKTDLISESLASRGKAVLQRSSRFLKESVGTRRKQQGITMKLDELKNLTSQFQESVDNITKQANDSLAMLRESPGGMREKGRKARELAAAANESAVKTLEDVLALSLRVFNTSEDLSRVNATVQETNDLLHNSTMTTLLAGRKMKDMEMQANLLLDRLKPLKTLEENLSRNLSEIKLLISRARKQAASIKVAVSADRDCIRAYQPQTSSTNYNTLILNVKTQEPDNLLFYLGSSSSSDFLAVEMRRGKVAFLWDLGSGSTRLEFPEVSINNNRWHSIYITRFGNMGSLSVKEASAAENPPVRTSKSPGPSKVLDINNSTLMFVGGLGGQIKKSPAVKVTHFKGCMGEAFLNGKSIGLWNYIEREGKCNGCFGSSQNEDSSFHFDGSGYAMVEKTLRPTVTQIVILFSTFSPNGLLFYLASNGTKDFLSIELVRGRVKVMVDLGSGPLTLMTDRRYNNGTWYKIAFQRNRKQGLLAVFDAYDTSDKETKQGETPGAASDLNRLEKDLIYVGGLPHSKAVRKGVSSRSYVGCIKNLEISRSTFDLLRNSYGVRKGCALEPIQSVSFLRGGYVEMPPKSLSPESSLLATFATKNSSGILLVALGKDAEEAGGAQAHVPFFSIMLLEGRIEVHVNSGDGTSLRKALLHAPTGSYSDGQEHSISLVRNRRVITIQVDENSPVEMKLGPLTEGKTIDISNLYIGGLPEDKATPMLKMRTSFHGCIKNVVLDAQLLDFTHATGSEQVELDTCLLAEEPMQSLHREHGELPPEPPTLPQPELCAVDTAPGYVAGAHQFGLSQNSHLVLPLNQSDVRKRLQVQLSIRTFASSGLIYYVAHQNQMDYATLQLQEGRLHFMFDLGKGRTKVSHPALLSDGKWHTVKTEYIKRKAFMTVDGQESPSVTVVGNATTLDVERKLYLGGLPSHYRARNIGTITHSIPACIGEIMVNGQQLDKDRPLSASAVDRCYVVAQEGTFFEGSGYAALVKEGYKVRLDLNITLEFRTTSKNGVLLGISSAKVDAIGLEIVDGKVLFHVNNGAGRITATYQPRAARALCDGKWHTLQAHKSKHRIVLTVDGNSVRAESPHTHSTSADTNDPIYVGGYPAHIKQNCLSSRASFRGCVRNLRLSRGSQVQSLDLSRAFDLQGVFPHSCPGPEP

SEQ ID NO: 3 - DNA, 30 bp, Artificial Sequence
  Olgionucleotide Primer
cgggatcctagagactgcatccgcgcctat

SEQ ID NO: 4 - DNA, 30 bp, Artificial Sequence
  Olgionucleotide Primer
cccaagctttactatctgcgtcacggtggg

SEQ ID NO: 5 - DNA, 33 bp, Artificial Sequence
  Olgionucleotide Primer
cgggatcctcagatagtaattctcttcagcacc

SEQ ID NO: 6 - DNA, 30 bp, Artificial Sequence
  Olgionucleotide Primer
cccaagcttggatgactcaggtgagagaga

SEQ ID NO: 7 - DNA, 28 bp, Artificial Sequence
  Olgionucleotide Primer
cgggatcctctgctggccacattcgcca

SEQ ID NO: 8 - DNA, 28 bp, Artificial Sequence
  Olgionucleotide Primer
cccaagcttcctcttccggacatcagac

SEQ ID NO: 9 - DNA, 30 bp, Artificial Sequence
  Olgionucleotide Primer
cgggatcctctccaggtgcagctgagcatt

SEQ ID NO: 10 - DNA, 30 bp, Artificial Sequence
  Olgionucleotide Primer
cccaagcttctgttggccattaaccatgat

SEQ ID NO: 11 - DNA, 30 bp, Artificial Sequence
  Olgionucleotide Primer
cgggatcctctggataaagacaggcccttg

SEQ ID NO: 12 - DNA, 32 bp, Artificial Sequence
  Olgionucleotide Primer
cccaagcttgggctcaggcccggggcaggaat